SEQUENCE LISTING (4 sequences)

SEQ ID NO: 1 - protein, 12 aa, Homo sapiens
VCNSPEPQYGGK

SEQ ID NO: 2 - protein, 14 aa, Homo sapiens
NALWHTGNTPGQVR

SEQ ID NO: 3 - protein, 12 aa, Homo sapiens
TRNMSACWQDGR

SEQ ID NO: 4 - protein, 7 aa, Homo sapiens
FYVVMWK

(19) United States
(12) Patent Application Publication
(10) Pub. No.: US 2020/0249235 A1
(43) Pub. Date: Aug. 6, 2020

(54) METHODS FOR DIAGNOSING PANCREATIC CANCER

(71) Applicants: THE TRUSTEES OF THE UNIVERSITY OF PENNSYLVANIA, Philadel, PA (US); MAYO FOUNDATION FOR MEDICAL EDUCATION AND RESEARCH, Rochester, MN (US)

(72) Inventors: Kenneth S. Zaret, Philadelphia, PA (US); Jungsun Kim, Portland, OR (US); Gloria M. Petersen, Rochester, MN (US); William R. Bamlet, Hayfield, MN (US)

(21) Appl. No.: 16/629,264

(22) PCT Filed: Jul. 6, 2018
(86) PCT No.: PCT/US2018/041108

§ 371 Date: Jan. 7, 2020

Related U.S. Application Data
(60) Provisional application No. 62/529,970, filed on Jul. 7, 2017.

(51) Int. Cl. G01N 33/574; C07K 14/435

(52) U.S. Cl. CPC G01N 33/57438; C07K 14/435

(57) ABSTRACT

The present disclosure relates to methods and kits for detecting pancreatic cancer (e.g., early stage pancreatic cancer, pre-cancerous lesions, and resectable pancreatic cancer) in a subject (e.g., human subject), including determining levels of THBS2 or a panel of THBS2/CA19-9 biomarkers in one or more biological sample obtained from the subject. The presently disclosed subject matter also relates to methods for determining THBS2 and CA19-9 cutoff values for use in the said methods and kit for detecting pancreatic cancer in a subject.

### CROSS REFERENCE TO RELATED APPLICATIONS

This application is a 35 U.S.C. § 371 national phase application from, and claims priority to International Application No. PCT/US2018/041108, filed Jul. 6, 2018, and published under PCT Article 21(2) in English, which claims priority to and the benefit of U.S. Provisional Application No. 62/529,970, filed Jul. 7, 2017, all of which applications are incorporated herein by reference in their entireties.

### STATEMENT REGARDING FEDERALLY SPONSORED RESEARCH OR DEVELOPMENT

This invention was made with government support under Grant Numbers R37GM36477, U01CA210138, and P50CA102701 awarded by the National Institutes of Health (NIH). The government has certain rights in the invention.

### BACKGROUND OF THE INVENTION

Pancreatic ductal adenocarcinoma (PDAC) is projected to become the second leading cause of cancer death in the United States by 2020 (1). The majority of PDAC patients are diagnosed at an advanced stage of disease and are not surgically resectable, contributing to a 7% overall 5-year survival rate (2). The lack of early diagnostics has made it challenging to develop therapeutics to slow or reverse PDAC (3). The CA19-9 serum marker is used to assess disease progression in PDAC patients (4, 5), but is not recommended for general screening (5, 6) because it is elevated in non-malignant pancreatic conditions such as chronic pancreatitis (7) and can produce false negatives in individuals who do not express Lewis blood group antigens (8). Other secreted markers have been reported for PDAC (9-12) including blood or urine proteins (13-15), exosomes (11), miRNAs (16), and epigenetic marks in circulating nucleosomes (17). However, challenges include lack of translation to the clinic, small sample sizes precluding statistical robustness, lack of blinded design, or inappropriate construction of datasets for development-to-validation (15-19). Certain biomarkers were discovered in advanced PDAC or cell lines that are not representative of earlier stages, when detection can be most relevant, although recent candidates have been tested or discovered in pre-diagnostic samples of PDAC (20-22). When agnostic biomarker panels are assessed in validation samples, the need to aggregate samples from multiple sources can hamper achieving statistical power (23).

Therefore, there remains a need to develop biomarkers facilitating early detection of pancreatic cancer in human subjects.

### SUMMARY OF THE INVENTION

The presently disclosed invention provides methods and kits for detecting pancreatic cancer (e.g., early stage pancreatic cancer and resectable pancreatic cancer) in a subject (e.g., human subject).

In one aspect, the invention includes a method for diagnosing pancreatic cancer or a predisposition for developing pancreatic cancer in a subject. The method comprises determining the concentration of both a THBS2 protein and a CA19-9 protein in a biological sample obtained from the subject, wherein an increase in the combination of values of the concentration of both THBS2 protein and CA19-9 protein in the biological sample from the subject, as compared with a concentration cutoff value for independently THBS2 protein or CA19-9 protein, is an indication that the subject has a pancreatic cancer or a predisposition for developing a pancreatic cancer, wherein when the pancreatic cancer or the predisposition for developing the pancreatic cancer is detected in the subject, an anti-cancer treatment is recommended for the subject.

In one aspect, the invention includes a method for determining the efficacy of an anti-cancer treatment for pancreatic cancer in a subject in need thereof. The method comprises determining the concentration of both a THBS2 protein and a CA19-9 protein in a biological sample obtained from the subject, wherein when the combination of values of the concentration of both THBS2 protein and CA19-9 protein in the biological sample from the subject is unchanged or lower as compared with a concentration cutoff value for independently THBS2 protein or CA19-9 protein, the treatment is efficacious, and when the treatment is not efficacious, an additional or a modified anti-cancer treatment is recommended for the subject.

In one aspect, the invention includes a method of determining whether a subject has pancreatic cancer. The method comprises (a) detecting a THBS2 measurement in one or more biological samples obtained from the subject; and (b) comparing the detected THBS2 measurement to at least one of a THBS2 measurement from a healthy subject and a THBS2 cutoff value, wherein an increase in the detected THBS2 measurement as compared to the healthy subject THBS2 measurement or the THBS2 cutoff value indicates that the subject has pancreatic cancer.

In one aspect, the invention includes a method of determining whether a subject has pancreatic cancer. The method comprises (a) detecting a THBS2 measurement and a CA19-9 measurement in one or more biological samples obtained from the subject; (b) comparing the detected THBS2 measurement to at least one of a THBS2 measurement from a healthy subject and a THBS2 cutoff value; and (c) comparing the detected CA19-9 measurement to a CA19-9 cutoff value, wherein an increase in the THBS2 measurement as compared to the healthy subject THBS2 measurement or the THBS2 cutoff value and/or an increase in the detected CA19-9 measurement as compared to the CA19-9 cutoff value indicates that the subject has pancreatic cancer.

In another aspect, the invention includes a kit for diagnosing whether a subject has pancreatic cancer. The kit comprises reagents useful for detecting THBS2 in one or more biological samples obtained from the subject.

In another aspect, the invention includes a kit for diagnosing whether a subject has pancreatic cancer. The kit comprises reagents useful for detecting THBS2 and CA19-9 in one or more biological samples obtained from the subject. In some embodiments, the kits of the invention comprise one or more of packaged probe and primer sets, arrays/microarrays, biomarker-specific antibodies or beads for detecting the THBS2 and/or CA19-9. In other embodiments, the kits of the invention comprise at least one monoclonal antibody or antigen-binding fragment thereof, or a polyclonal antibody or antigen-binding fragment thereof, for detecting the THBS2 and/or CA19-9. In other embodiments, the kits of the invention further comprise an instruction describing that an increase in the level of the THBS2 as compared to a THBS2 cutoff value indicates that the subject has pancreatic cancer. In other embodiments, the kits of the invention, further comprise an instruction describing that an increase in the level of the THBS2 as compared to a THBS2 cutoff value and/or an increase in the level of the CA19-9 as compared to a CA19-9 cutoff value indicates that the subject has pancreatic cancer.

In yet another aspect, the invention includes a method for determining a cutoff value of THBS2 for diagnosis of pancreatic cancer in a subject. The method comprises (a) measuring the distribution of THBS2 values in a group of healthy subjects; (b) selecting a THBS2 value wherein the selected THBS2 value has a false positive rate of between about 0 to about 5%; and (c) measuring the sensitivity value and specificity value of the selected THBS2 value in detecting pancreatic cancer in a group of subjects having pancreatic cancer, wherein the sensitivity value of at least about 50% and the specificity value of at least about 90% indicate the selected THBS2 value is a cutoff value of THBS2 for diagnosis of pancreatic cancer in a subject. In some embodiments, the THBS2 cutoff value, as used in the methods and kits of the invention, is determined by the method for determining a cutoff listed above herein.

In another aspect, the invention includes a method of managing a subject suspected of having pancreatic cancer. The method comprises (a) detecting a THBS2 measurement in one or more biological samples from the subject; and (b) comparing the detected THBS2 measurement to at least one of a THBS2 measurement from a healthy subject and/or a THBS2 cutoff value, wherein if an increase in the detected THBS2 measurement as compared to the healthy subject THBS2 measurement or the THBS2 cutoff value is detected, an imaging evaluation is performed to detect pancreatic cancer, wherein if the pancreatic cancer is confirmed by the imaging evaluation, a biopsy of the pancreatic cancer is performed on the subject, followed by an anti-cancer treatment of the subject.

In a further aspect, the invention includes a method of managing a subject suspected of having pancreatic cancer. The method comprises (a) detecting a THBS2 measurement in one or more biological samples obtained from the subject; and (b) comparing the detected THBS2 measurement to at least one of a THBS2 measurement from a healthy subject and/or a THBS2 cutoff value, wherein if an increase in the detected THBS2 measurement as compared to the healthy subject THBS2 measurement or the THBS2 cutoff value is not detected, follow-up screening/surveillance is performed to monitor the subject for early stage pancreatic cancer.

In yet further aspect, the invention includes a method of managing a subject suspected of having pancreatic cancer. The method comprises (a) detecting a THBS2 measurement and a CA19-9 measurement in the one or more biological samples obtained from the subject; (b) comparing the detected THBS2 measurement to at least one of a THBS2 measurement from a healthy subject and a THBS2 cutoff value; and (c) comparing the detected CA19-9 measurement to a CA19-9 cutoff value, wherein if an increase in the THBS2 measurement as compared to the healthy subject THBS2 measurement or the THBS2 cutoff value and/or an increase in the detected CA19-9 measurement as compared to the CA19-9 cutoff value is detected, an imaging evaluation is performed to detect pancreatic cancer, wherein if the pancreatic cancer is confirmed by the imaging evaluation, a biopsy of the pancreatic cancer is performed on the subject, followed by an anti-cancer treatment of the subject.

In another aspect, the invention includes a method of managing a subject suspected of having pancreatic cancer. The method comprises (a) detecting a THBS2 measurement and a CA19-9 measurement in the one or more biological samples obtained from the subject; (b) comparing the detected THBS2 measurement to at least one of a THBS2 measurement from a healthy subject and a THBS2 cutoff value; and (c) comparing the detected CA19-9 measurement to a CA19-9 cutoff value, wherein if neither an increase in the THBS2 measurement as compared to the healthy subject THBS2 measurement or the THBS2 cutoff value nor an increase in the detected CA19-9 measurement as compared to the CA19-9 cutoff value is detected, a follow-up screening/surveillance is performed to monitor the subject for early stage pancreatic cancer.

In another aspect, the invention includes a method for treating pancreatic cancer in a subject. The method comprises (a) detecting a combination of values in the concentration of both THBS2 protein and CA19-9 protein, in a biological sample obtained from the subject, (b) comparing the combination of values in the concentration of both THBS2 protein and CA19-9 protein in the biological sample, with a concentration cutoff value for independently THBS2 protein or CA19-9 protein, and (c) when the combination of values in the concentration of both THBS2 protein and CA19-9 protein in the biological sample is higher than the concentration cutoff value for independently THBS2 protein or CA19-9 protein, the subject is administered a chemotherapy, a radiation therapy, an immunotherapy, a gene therapy, a biological modifier therapy, or a cancer vaccine therapy to the subject, wherein the number of pancreatic cancer cells within the subject is reduced.

In another aspect, the invention includes a method for treating pancreatic cancer in a subject. The method comprises (a) detecting a combination of values in the concentration of both THBS2 protein and CA19-9 protein in a biological sample obtained from the subject, (b) comparing the combination of values in the concentration of both THBS2 protein and CA19-9 protein in the biological sample, with a concentration cutoff value for independently THBS2 protein or CA19-9 protein, and (c) when the combination of values in the concentration of both THBS2 protein and CA19-9 protein in the biological sample is higher than the concentration cutoff value for independently THBS2 protein or CA19-9 protein, the pancreatic cancer cells are surgically removed from the subject.

In another aspect, the invention includes a method for treating pancreatic cancer in a subject. The method comprises administering a chemotherapy, a radiation therapy, an immunotherapy, a gene therapy, a biological modifier therapy, or a cancer vaccine therapy to a subject identified as having pancreatic cancer and a combination of values in the concentration of both THBS2 protein and CA19-9 protein in a biological sample obtained from the subject, wherein the combination of values in the concentration of both THBS2 protein and CA19-9 protein in the biological sample is higher than a concentration cutoff value for independently THBS2 protein or CA19-9 protein, and wherein the number of pancreatic cancer cells within the subject is reduced.

In another aspect, the invention includes a method for treating pancreatic cancer in a subject. The method comprises surgically removing pancreatic cancer cells from a subject identified as having pancreatic cancer and a combination of values in the concentration of both THBS2 protein and CA19-9 protein in a biological sample obtained from the subject, wherein the combination of values in the concentration of both THBS2 protein and CA19-9 protein in the biological sample is higher than a concentration cutoff value for independently THBS2 protein or CA19-9 protein.

In various aspects of embodiments of the above aspects or any other aspect of the invention delineated herein, the subject is a human.

In some embodiments, the pancreatic cancer is an early stage pancreatic cancer or a resectable pancreatic cancer. In other embodiments, the pancreatic cancer is a pancreatic ductal adenocarcinoma (PDAC). In other embodiments, when pancreatic cancer is detected in the subject, an anti-cancer treatment is recommended for the subject.

In some embodiments, the anti-cancer treatment comprises at least one selected from the group consisting of surgical treatment, chemotherapy, radiation therapy, immunotherapy, gene therapy, biological modifier therapy, and cancer vaccine therapy.

In some embodiments, the biological sample is selected from the group consisting of a blood sample, a serum sample, a plasma sample, a stool sample, a urine sample, a pancreatic cyst fluid sample, and a tissue sample.

In some embodiments, the THBS2 comprises a transcribed polynucleotide of THBS2 or portion thereof.

In some embodiments, THBS2 is a THBS2 protein and CA19-9 is a CA19-9 protein. In other embodiments, the level or the concentration of both THBS2 protein and CA19-9 protein is detected using a reagent that specifically binds to THBS2 protein and CA19-9 protein, respectively. In some embodiments, the level or concentration of the THBS2 protein and/or CA19-9 protein is detected by enzyme-linked immunosorbent assay (ELISA). In some embodiments, the reagent is a monoclonal antibody or antigen-binding fragment thereof, or a polyclonal antibody or antigen-binding fragment thereof.

In other embodiments, the THBS2 cutoff value is a THBS2 protein cutoff value of between about 30 to about 50 ng/ml. In yet other embodiments, the THBS2 protein cutoff value is about 42 ng/ml. In other embodiments, the CA19-9 cutoff value is between about 30 to about 1000 U/ml. In yet other embodiments, the CA19-9 cutoff value is a CA19-9 protein cutoff value of about 55 U/ml.

In some embodiments, the THBS2 protein comprises an amino acid sequence of SEQ ID NO: 1.

In some embodiments, the specificity of the combination of values is at least 90%. In other embodiments, the specificity of the combination of values is at least 95%. In yet other embodiments, the specificity is about 99%.

In some embodiments, the sensitivity value is about 50%. In some embodiments, the sensitivity of the combination of values is at least 80%. In other embodiments, the sensitivity of the combination of values is at least 85%.

In some embodiments, the false positive rate is about 1%.

### BRIEF DESCRIPTION OF THE DRAWINGS

FIGS. 1A-1B. Phase 1 validation studies and THBS2 expression analysis in diverse human cancers. (1A) AUC analysis of blinded ELISA data for MMP2, MMP10, and THBS2 on plasma samples from 10 PDAC patients at designated stages compared to 10 healthy controls. (1B) Boxplots of THBS2 mRNA expression measured in various tumors (sample sizes in parentheses), assessed by RNA-seq. Tumors are sorted in order of decreasing median patient expression value. Of the TGCA pancreatic cancer samples (n=179) based upon the TCGA clinical manifest, only PDAC (n=134) was analyzed. All expression values are log2 (RSEM values=1) transformed.

FIGS. 2A-2G. The concentrations of THBS2 and CA19-9 in all stage PDAC cases vs healthy primary care controls in Phase 2a and Phase 2b cohorts. (2A & 2D) Scatter plots of THBS2 concentrations in all stage PDAC cases vs controls for Phase 2a and 2b respectively. (2B) Area under ROC curve (AUC) with a bootstrapped (1000 repetitions) 95% CI. (2C & 2E) ROC curves of THBS2, CA19-9, and THBS2+CA19-9 in all stage PDAC cases vs. controls for Phase 2a (PDAC n=81, controls n=80) and 2b (PDAC n=197, controls n=140) respectively. P values shown. (2F & 2G) Scatter plots of THBS2 and CA19-9 in all stage PDAC cases vs. healthy primary care controls for Phase 2a and Phase 2b, respectively.

FIGS. 3A-3E. THBS2 and CA19-9 in all PDAC cases versus benign pancreatic diseases. (3A) Area under ROC curve (AUC) with a bootstrapped (1000 repetitions) 95% CI for all stages PDAC versus patients with benign pancreatic diseases (Phases 2a and 2b). P values shown. Subsequent panels display ROC curves of THBS2, CA19-9, and THBS2+CA19-9 generated from Phase 2b data for PDAC (n=197) vs. pancreatitis (n=55, 3B), PDAC vs. IPMN (n=115, 3C), PDAC vs. PNET (n=30, 3D), and PNET (n=30) vs. healthy controls (n=140, 3E).

FIGS. 4A-4K. Expression of THBS2 in human PanIN and PDAC tissues. (4A & 4B) Immunohistochemistry for THBS2 was performed on incidental PanIN I-II tissue derived from the head and neck of a pancreas from a pancreatic periampullary cancer patient at the Fox Chase Cancer Center, as covered under IRB 09-801 to K. Zaret, with two different antibodies (Panel 4A, Origene TA590658; Panel 4B, Santa Cruz sc-7655). The arrows indicate positively stained PanIN2s; dotted arrow indicates weak or negative staining of PanIN1. THBS2 expression, designated by arrows, was also confirmed in stage II PDAC (4C-4E) and stage III (4F-4K) pancreatic cancer tissue arrays (US Biomax, PA1002). Competitive assays were performed for antibody 2 (sc-7655) by pre-incubating the antibody with a 10-fold excess of antigen peptide (4E, 4H, 4K), to confirm target specificity). Brown color indicates THBS2 staining and blue color indicates hematoxylin nuclear staining. THBS2 was detected in the epithelial cells of non-invasive lesions (PanINs and IPMNs) and poorly differentiated PDAC as well as fibroblastic cells in the invasive PDAC stages (See Table 10 and FIGS. 10A-10E).

FIGS. 5A-5B. Validation of lack of THBS1 cross-reactivity of antibodies used in the THBS2 ELISA kit by Western Blot. (5A) SDS-PAGE confirmation of expected relative sizes of recombinant THBS1, THBS2, THBS3, and THBS4 proteins obtained from Bio-Techne, Inc. The company had validated the specificity of the THBS2 ELISA kit with these recombinant proteins. Because of prior claims that THBS1 is down-regulated in PDAC (see text), the cross-reactivity of THBS1 against the detection and capture antibodies in the THBS2 ELISA kit in duplicate experiments (Exp1 and Exp2) was assessed. (5B) ELISA detection and capture antibodies for THBS2 are competed by THBS2, not by THBS1. (i) Samples (0.15 nM) of goat polyclonal anti-THBS2 detection antibody were used with no competitor or with a 100-fold molar excess of recombinant proteins THBS2 or THBS1 for 30 min at room temperature. The reactions were applied to Western blot membranes with 2 or 10 ng THBS2 in separate lanes. The excess THBS2 competes for the signal while the excess THBS1 does not. To demonstrate that the membrane for the THBS2 competition did have THBS2 protein, the first antibody reaction was stripped and the blot was re-probed (arrow) with the THBS2 antibody without competitor. (ii) Samples (3 nM) of mouse monoclonal anti-THBS2 capture antibody were used with no competitor or with a 10-fold molar excess of recombinant proteins THBS2 or THBS1 for 30 min at room temperature. All THBS2 proteins were run in a gel and transferred into the same membrane. The membrane was blocked and divided into three pieces before applying the mixture of detection antibody and competitors. The reactions were applied to Western blot membranes with 10 or 50 ng THBS2 in separate lanes. The excess THBS2 competes for the signal while the excess THBS1 does not. To demonstrate that the membrane for the THBS2 competition did have THBS2 protein, the blot was stripped and stained with silver. Arrows point to recombinant THBS2 evident on the blot (signals were saturated at 10 ng). Brackets indicate the marker bands that spilled over from neighboring lanes. Note that the monoclonal capture antibody has less sensitivity than the polyclonal detection antibody.

FIGS. 6A-6B. Validation of lack of THBS1 cross-reactivity or interference in the THBS2 ELISA assay. (6A) Presence of excess THBS1 has no effect on ELISA detection of recombinant THBS2 proteins. To determine whether presence of THBS1 interferes with the THBS2 ELISA kit, 200 ng/ml of recombinant THBS1 protein was spiked into various concentrations of recombinant THBS2 proteins (0 ng/ml to 20 ng/ml) and subjected to THBS2 ELISA assays. Grey bars indicate THBS2 protein alone and black bars indicate THBS2 proteins spiked with THBS1. The differences in THBS2 detection are negligible (less than 5% CV). (6B) Presence of excess THBS1 has no effect on ELISA detection of THBS2 in human plasma. ELISA assays were performed on Phase 2b plasma samples randomly picked to exhibit a range of THBS2 concentrations, along with human normal pooled plasma, with and without 200 ng/ml THBS1 protein. Grey bars show the plasma alone and black bars show the plasma with a 200 ng/ml of THBS1. The differences in THBS2 concentration in Phase 2b plasmas between absence and presence of THBS1 protein (200 ng/ml) were less than 5% CV, while the very low concentrations in the commercial normal pooled plasma exhibited a 10% CV). Primary data are shown at bottom.

FIG. 7. Reproducibility of the ELISA assay for THBS2. The plot shows primary ELISA data in nanograms THBS2 per ml of plasma for a set of Phase 2a samples. The signals were determined from a standard curve as described in the Materials and Methods. The same samples were assessed once in 2014 and twice in 2015, using different lots for each assay. The two assays in 2015 included a commercial plasma sample mixed from normal, healthy individuals (*denoted “control”).

FIGS. 8A-8B. Distribution of THBS2 values in Phase 2 samples. Data for Phase 2a (8A) and Phase 2b (8B) plasma samples in ELISA were rank-ordered and divided into 7 groups based upon THBS2 protein concentrations. The frequency of samples in each group is shown.

FIGS. 9A-9B. Cross-validation tests, performed one year apart, of THBS2 concentrations in the same set of plasmas as determined in different laboratories. A subset of Phase 2b samples were cross-validated in a blinded fashion, in an independent lab that was provided only the relevant methods section of this paper and the manufacturer's instructions for methodology. (9A) Scatterplot of original THBS2 values versus cross-validated THBS2 values, as determined one year later with a different lot number of reagents, in subset of Phase 2b plasma samples (n=38). The X-axis indicates the THBS2 values originally obtained in 2015 and Y-axis indicates the THBS2 values cross-validated by an independent lab in 2016. The Pearson correlation coefficient is 0.95 and the Spearman coefficient is 0.968, indicating a strong correlation between the original and validated assays. (9B) Scatterplot of original THBS2 versus cross-validated THBS2 values rescaled by commercial human normal pooled plasma. The average THBS2 value in the normal pooled plasma control in the original assay, where this set point was derived, was 17 ng/ml and the average THBS2 value in the normal pooled plasma control in the cross-validated samples was 13.25. Thus the data was re-plotted, as shown, by dividing the cross-validation samples by the scaling factor as described in the text. The re-scaled THBS2 values in cross-validation had a negligible effect on the correlation coefficient, indicating a robust assay.

FIGS. 10A-10E. Representative immunohistochemistry (IHC) images of THBS2 in human normal pancreas, pancreatitis, and PDAC tissue. Two “IHC quality” THBS2 antibodies were tested: Ab#1 (rabbit polyclonal THBS2 antibody, dilution 1:100, TA590658, Origene) and Ab#2 (Goat polyclonal THBS2 antibody, dilution 1:25, sc-7655, Santa Cruz). Red arrows designate positive cells. (10A) IHC in human normal pancreas. THBS2 was detected in acinar and ductal compartment but not in stroma with Ab#1. However, THBS2 was barely detected in the acinar compartment and not in ducts or stroma with Ab#2. (10B) IHC in human pancreatitis. THBS2 was detected in incidental PanINs in pancreatitis and acinar cells but not in stroma cells with both antibodies. (10C) IHC in cancerous IPMN stage T3, pNo, pMX, Grade 1. THBS2 was detected in cancerous IPMN but not in stromal cells with both antibodies. (10D) IHC in PDAC stage 2b. THBS2 was detected in PDAC epithelial cells and weakly labeled in acinar cells, but not in stroma cells with both antibodies. (10E) IHC in recurrent T2N1Mx PDAC. THBS2 was detected in both cancer epithelial and stroma cells with both antibodies. Brown colors indicate the compartments labeled with THBS2 in the left panel.

### DETAILED DESCRIPTION

The presently disclosed subject matter relates to methods and kits for detecting and treating pancreatic cancer (e.g., early stage pancreatic cancer and resectable pancreatic cancer) in a subject (e.g., human subject), including determining levels of THBS2 or a panel of THBS2/CA19-9 biomarkers in a biological sample obtained from the subject. The presently disclosed subject matter also relates to methods for determining THBS2 cutoff values for use in the said methods and kit for detecting pancreatic cancer in a subject.

For purposes of clarity of disclosure and not by way of limitation, the detailed description is divided into the following subsections:

5.1. Definitions;

5.2. THBS2 or a THBS2/CA19-9 panel as biomarkers for pancreatic cancer

5.3. Method for determining cutoff values of THBS2

5.4. Biomarker detection

5.4. Kit

### 5.1. Definitions

The terms used in this specification generally have their ordinary meanings in the art, within the context of this invention and in the specific context where each term is used. Certain terms are discussed below, or elsewhere in the specification, to provide additional guidance to the practitioner in describing the compositions and methods of the invention and how to make and use them.

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, e.g., up to 10%, up to 5%, or up to 1% of a given value. Alternatively, particularly with respect to biological systems or processes, the term can mean within an order of magnitude, e.g., within 5-fold, or within 2-fold, of a value.

As used herein, the use of the word “a” or “an” when used in conjunction with the term “comprising,” “comprise,” “includes” or “including” in the claims and/or the specification can mean “one,” but it is also consistent with the meaning of “one or more,” “at least one,” and “one or more than one.” Certain embodiments of the present disclosure can consist of or consist essentially of one or more elements, method steps and/or methods of the invention. It is contemplated that any method or composition described herein can be implemented with respect to any other method or composition described herein.

As used herein, the term “biomarker” refers to a marker (e.g., an expressed gene, including mRNA and/or protein) that allows detection of a disease in an individual, including detection of disease in its early stages. Biomarkers, as used herein, include nucleic acid and/or protein markers. In certain non-limiting embodiments, a biomarker is a released and/or secreted protein that can be detected in a biological sample of a subject. In certain embodiments, the expression level of a biomarker as determined by mRNA and/or protein levels in tissue or biological sample from an individual to be tested is compared with respective levels in normal tissue or biological sample from the same individual or another healthy individual. In certain embodiments, the expression level of a biomarker as determined by mRNA and/or protein levels in tissue or biological sample from an individual to be tested is compared with a predetermined cutoff value established in a group of healthy individuals and pancreatic cancer patients. In certain embodiments, an increase in the expression level of the biomarker of a biomarker as determined by mRNA and/or protein levels in tissue or biological sample from an individual to be tested as comparing with respective levels in normal tissue or biological sample from the same individual or another healthy individual indicates that the individual has pancreatic cancer. In certain embodiments, an increase in the expression level of a biomarker as determined by mRNA and/or protein levels in tissue or biological sample from an individual to be tested as comparing with a predetermined cutoff value of the biomarker indicates that the individual has pancreatic cancer.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCRTM, and the like, and by synthetic means. In some embodiments, a nucleic acid sequence is considered to have at least 95%, 96%, 97%, 98%, or 99% identity or homology to any nucleic acid sequence disclosed herein.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof. In some embodiments, an amino acid sequence is considered to have at 95%, 96%, 97%, 98%, or 99% identity or homology to any amino acid sequence described herein.

As used herein, the term “combination of” or “combination of values” refers to the summation of the amount of the biomarkers THBS2 and CA19-9 detected in a biological sample from a subject of interest (e.g. a human patient) which can be the protein concentration level of these biomarkers (e.g. ng/ml or U/ml). In some aspects of the invention, the combination of values of the concentration of both THBS2 protein and CA19-9 protein is synergetic and is compared with a concentration cutoff value for independently THBS2 protein or CA19-9 protein. As non-limiting examples, this comparison can be useful for diagnosing pancreatic cancer or a predisposition for developing pancreatic cancer or determining the efficacy of an anti-cancer treatment for pancreatic cancer in a subject. In some embodiments, the combination of values of the concentration of both THBS2 protein and CA19-9 protein is greater than zero. In other embodiments, the THBS2 protein concentration is greater than zero. In yet other embodiments, the CA19-9 concentration is zero or is greater than zero.

As used herein, the term “sensitivity” refers to the proportion of positives that are correctly identified by the biomarker (e.g., THBS2, or THBS2/CA19-9 panel) as such (i.e., subjects identified as having pancreatic cancer truly have pancreatic cancer). It also refers to the extent to which true positives are not missed/overlooked.

As used herein, the term “specificity” refers to the proportion of negatives that are correctly identified by the biomarker (e.g., THBS2, or THBS2/CA19-9 panel) as such (i.e., the percentage of healthy subjects who are correctly identified as not having the pancreatic cancer).

As used herein “cutoff value” refers to the dividing point on measuring scale where the test results are divided into different categories (e.g., indicating a subject having pancreatic cancer or not having pancreatic cancer). In certain embodiments, the cutoff value is THBS2 cutoff value. In certain embodiments, the cutoff value is a CA19-9 cutoff value. In certain embodiments, the cutoff value is a combination of THBS2 cutoff value and/or CA19-9 cutoff value.

As used herein, the term “false positive rate” refers to the percentage of healthy subjects who incorrectly receive a positive test result (i.e., the percentage of healthy subjects who are identified as having pancreatic cancer by measuring of THBS2 or THBS2/CA19-9 panel).

As used herein, the term “biological sample” refers to a sample of biological material obtained from a subject, e.g., a human subject, including tissue, a tissue sample, a cell sample, a tumor sample, a stool sample and a biological fluid, e.g., plasma, serum, blood, urine, lymphatic fluid, ascites, pancreatic cyst fluid and a nipple aspirate. In certain embodiments, the presence of one or more biomarkers is determined in a peripheral blood sample obtained from a subject. In certain embodiments, the presence of one or more biomarkers is detected in a stool sample obtained from a subject. In certain embodiments, the presence of one or more biomarkers is detected in pancreatic cyst fluid obtained from a subject. In certain embodiments, the presence of one or more biomarkers is detected in one or more plasma samples obtained from a subject.

The term “pancreatic cancer” as described herein refers to any type of cancerous or precancerous tissues arising from normal tissues of the pancreas, including, but not limited to, pre-cancerous lesion, PanIN lesions, pancreatic ductal adenocarcinoma or pancreatic adenocarcinoma. Other types of pancreatic tumors include acinar-cell carcinoma, serous cystadenoma and pancreatic endocrine tumors. In certain embodiments, the biomarkers of the present disclosure can be used to detect cancers such as biliary cancer and liver cancer. In certain embodiments, the pancreatic cancer is a pre-cancerous lesion that develops into pancreatic cancer later.

As used herein “early stage pancreatic cancer” refers to a subset of pancreatic cancers that have not spread to large blood vessels or distant organs such as the liver or lungs. For example and not by way of limitation, Stages I and IIA of pancreatic cancer are considered as early stage pancreatic cancer in the 6th Edition American Joint Committee on Cancer (“AJCC”) Pancreatic Cancer Staging System.

As used herein “resectable pancreatic cancer” refers to a subset of pancreatic cancers that can be surgically excised. For example and not by way of limitation, stages I, IIA and IIB of pancreatic cancer are resectable tumors in the 6th Edition AJCC Pancreatic Cancer Staging System.

An “individual” or “subject” herein is a vertebrate, such as a human or non-human animal, for example, a mammal. Mammals include, but are not limited to, humans, primates, farm animals, sport animals, rodents and pets. Non-limiting examples of non-human animal subjects include rodents such as mice, rats, hamsters, and guinea pigs; rabbits; dogs; cats; sheep; pigs; goats; cattle; horses; and non-human primates such as apes and monkeys.

As used herein, the term “treating” or “treatment” refers to clinical intervention in an attempt to alter the disease course of the individual or cell being treated, and can be performed either for prophylaxis or during the course of clinical pathology. Therapeutic effects of treatment include, without limitation, preventing occurrence or recurrence of disease, alleviation of symptoms, and diminishment of any direct or indirect pathological consequences of the disease, preventing metastases, decreasing the rate of disease progression, amelioration or palliation of the disease state, and remission or improved prognosis. By preventing progression of a disease or disorder, a treatment can prevent deterioration due to a disorder in an affected or diagnosed subject or a subject suspected of having the disorder, but also a treatment may prevent the onset of the disorder or a symptom of the disorder in a subject at risk for the disorder or suspected of having the disorder.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### 5.2. THBS2 or a THBS2/CA19-9 Panel as Biomarkers for Pancreatic Cancer

THBS2 is a glycoprotein that is thought to be an angiogenesis inhibitor, and mutation of the mouse TSP-2 gene elevates cancer susceptibility (43). It has been found that THBS2 antigen is expressed in normal pancreas cells, yet the baseline concentration of THBS2 is very low in normal human plasma, as measured by both mass spectrometry and ELISA, and is elevated in PDAC. It has been found that THBS2 antigen is robustly expressed in pancreatic cancer cells, concordant with the poor vascularization associated with PDAC.

In one aspect, the invention includes a method for diagnosing pancreatic cancer or a predisposition for developing pancreatic cancer in a subject. The method of the invention comprises determining the concentration of both a THBS2 protein and a CA19-9 protein in a biological sample obtained from the subject, wherein an increase in the combination of values of the concentration of both THBS2 protein and CA19-9 protein in the biological sample from the subject, as compared with a concentration cutoff value for independently THBS2 protein or CA19-9 protein, is an indication that the subject has a pancreatic cancer or a predisposition for developing a pancreatic cancer, wherein when the pancreatic cancer or the predisposition for developing the pancreatic cancer.

In some embodiments, when the pancreatic cancer or the predisposition for developing the pancreatic cancer is detected in the subject, an anti-cancer treatment, is recommended for the subject.

In another aspect, the invention includes a method for determining the efficacy of an anti-cancer treatment against pancreatic cancer in a subject in need thereof. The method comprises determining the concentration of both a THBS2 protein and a CA19-9 protein in a biological sample obtained from the subject, wherein when the combination of values of the concentration of both THBS2 protein and CA19-9 protein in the biological sample from the subject is unchanged or lower as compared with a concentration cutoff value for independently THBS2 protein or CA19-9 protein, the treatment is efficacious, and when the treatment is not efficacious, an additional or a modified anti-cancer treatment is recommended for the subject.

In certain embodiments, the presently disclosed subject matter provides a method of determining whether a subject has pancreatic cancer, comprising: obtaining one or more biological samples from the subject; and detecting THBS2 in the one or more biological samples, wherein an increase in the level of the THBS2 as compared to a healthy subject indicates that the subject has pancreatic cancer. In certain embodiments, an increase in the level of the THBS2 with a predetermined THBS2 cutoff value indicates that the subject has pancreatic cancer.

In certain embodiments, a THBS2/CA19-9 panel is used to determine whether a subject has a pancreatic cancer. CA19-9 is not expressed in certain pancreatic patients, e.g., patients being Lewis antigen negative. The THBS2/CA19-9 panel for detecting pancreatic cancer expands the specificity and sensitivity of CA19-9 or THBS2 alone in detecting pancreatic cancer. In certain embodiments, the presently disclosed subject matter provides for a method of determining whether a subject has pancreatic cancer, comprising: obtaining one or more biological samples from the subject; and detecting THBS2 and CA19-9 in the one or more biological samples, wherein an increase in the level of the THBS2 as compared to a healthy subject and/or an increase in the level of the CA19-9 as compared to a CA19-9 cutoff value indicates that the subject has pancreatic cancer. In certain embodiments, an increase in the level of the THBS2 as compared to a THBS2 cut-off value and/or an increase in the level of the CA19-9 as compared to a CA19-9 cutoff value indicates that the subject has pancreatic cancer

In certain embodiments the CA19-9 cutoff value is a CA19-9 protein cutoff value. In certain embodiments, CA19-9 protein cutoff value is between about 30 to about 1000 U/ml. In other embodiments, CA19-9 protein cutoff value is between about 30 to more than 1000 U/ml, between about 30 to about 1500 U/ml, between about 30 to about 2000 U/ml, between about 30 to about 2500 U/ml, between about 30 to about 3000 U/ml, or between about 30 to more than 3000 U/ml. In yet other embodiments, the CA19-9 protein cutoff value is between about 40 to 45 U/ml, between about 45 to 55 U/ml, between about 50 to 55 U/ml, or between about 55 to 60 U/ml. In certain embodiments, the CA19-9 protein cutoff value is about 55 U/ml.

In certain embodiments the THBS2 cutoff value is a THBS2 protein cutoff value. In certain embodiments, the THBS2 cutoff value is between about 20 to about 100 ng/ml, between about 20 to about 25 ng/ml, between about 25 to about 30 ng/ml, between about 30 to about 35 ng/ml, between about 35 to about 40 ng/ml between about 40 to about 45 ng/ml, between about 45 to about 50 ng/ml, between about 50 to about 55 ng/ml between about 55 to about 60 ng/ml, between about 60 to about 65 ng/ml between about 65 to about 70 ng/ml, between about 70 to about 75 ng/ml, between about 75 to about 80 ng/ml, between about 85 to about 90 ng/ml, between about 90 to about 95 ng/ml, or between about 95 to about 100 ng/ml. In certain embodiments, the THBS2 cutoff value is about 36 ng/ml, about 37 ng/ml, about 38 ng/ml, about 39 ng/ml, about 40 ng/ml, about 41 ng/ml, about 42 ng/ml, about 43 ng/ml, about 44 ng/ml, or about 45 ng/ml. In certain embodiments, the THBS2 cutoff value is about 42 ng/ml.

In certain embodiments, THBS2 protein comprises an amino acid sequence of SEQ ID NO: 1.

In certain embodiments, the biological sample can be a blood sample (e.g., a plasma or serum sample), or a feces sample (e.g., a stool sample). In certain embodiments, the biological sample can be a tissue sample (e.g., a pancreatic cyst fluid). The step of collecting a biological sample can be carried out either directly or indirectly by any suitable technique. For example, a blood sample from a subject can be carried out by phlebotomy or any other suitable technique, with the blood sample processed further to provide a serum sample or other suitable blood fraction, e.g., plasma, for use in the methods of the presently disclosed subject matter.

Currently, there are no screening tests that can lead to actionable management for early stage pancreatic cancer in human subjects. The information provided by the methods for detecting pancreatic cancer using THBS2 or THBS2/CA19-9 biomarkers as described herein can be the basis for managing a subject. In certain embodiments, the method of managing the subject is an invasive diagnostic evaluation. In certain embodiments, if the subject is determined to have pancreatic cancer based on the method of determining whether a subject has pancreatic cancer as described herein, the method of managing includes imaging evaluation to detect the pancreatic cancer. Non-limiting examples of imaging evaluation are magnetic resonance imaging, computed tomography, and/or endoscopic ultrasound of the pancreas. If the results of the imaging evaluation support the presence of pancreatic cancer, a biopsy of the pancreatic cancer mass followed by a surgical resection and/or a chemotherapy can be performed. In certain embodiments, if the subject is determined to not have pancreatic cancer based on the method of determining whether a subject has pancreatic cancer as described herein, the method of managing can be a follow-up and future screening/surveillance for pancreatic cancer. In certain embodiments, the intervals of the follow-up and future screening/surveillance is based on additional predictors and/or risk factors. Non-limiting examples of predictors and risk factors are weight loss and genetic risk factors. In certain embodiments, the follow-up and future screening/surveillance include the method of determining whether a subject has pancreatic cancer as described herein.

In certain embodiments, the information provided by the methods described herein can be used by the physician in determining the most effective course of treatment (e.g., preventative or therapeutic). A course of treatment refers to the measures taken for a patient after the assessment of increased risk for development of pancreatic cancer is made. For example, when a subject is identified to have an increased risk of developing cancer, the physician can determine whether frequent monitoring for biomarker detection is required as a prophylactic measure. Also, when the subject is determined to have pancreatic cancer (e.g., based on the presence of one or more biomarkers in a biological sample from a subject), it can be advantageous to follow such detection with a biopsy, surgical treatment, chemotherapy, radiation, immunotherapy, biological modifier therapy, gene therapy, vaccines and the like, or adjust the span of time during which the patient is treated.

The invention also includes materials and methods for treating a patient (e.g., a human patient) identified as having pancreatic cancer as described herein. For example, a human identified as having pancreatic cancer based, at least in part, on the presence of an elevated level of THBS2 polypeptide expression and/or an elevated level of CA19-9 polypeptide expression as compared with a concentration cutoff value for independently THBS2 protein or CA19-9 protein, would be referred for pancreatic imaging and treated with an anti-cancer therapy.

The optimal dosage and treatment regime for a particular patient can readily be determined by a person skilled in the art by monitoring the patient for signs of disease and adjusting the treatment accordingly. A person skilled in the art can recommend any appropriate anti-cancer treatment known in the art at the time or the invention or thereafter.

In non-limiting examples, a patient can be administered one or more chemotherapies, one or more radiation therapies, one or more immunotherapies, one or more biological modifier therapies, one or more gene therapies, and/or one or more anti-cancer vaccine therapies to reduce the number of pancreatic cancer cells present within the patient. In some cases, a patient (e.g. human) identified as having pancreatic cancer based, at least in part, on the presence of an elevated level of THBS2 polypeptide expression and/or an elevated level of CA19-9 polypeptide expression can be treated surgically to remove pancreatic cancer cells present within the patient. In some cases, a combination of surgery to remove pancreatic cancer cells present within the patient and (a) one or more chemotherapies, (b) one or more radiation therapies, (c) one or more immunotherapies, (c) one or more biological modifier therapies, (d) one or more gene therapies, and/or (e) one or more anti-cancer vaccine therapies can be used to treat a patient (e.g., a human patient) identified as having pancreatic cancer as described herein.

Examples of chemotherapies that can be used to treat a patient (e.g., a human patient) identified as having pancreatic cancer as described herein include, without limitation, gemcitabine, 5-FU/leucovorin, capecitabine, FOLFIRINOX, erlotinib, paclitaxel, and the like. Examples of radiation therapies that can be used to treat a patient (e.g., a human patient) identified as having pancreatic cancer as described herein include, without limitation, radiation, chemoradiation, stereotactic body radiotherapy, proton beam, and the like. Examples of biological modifier therapies that can be used to treat a patient (e.g., a human patient) identified as having pancreatic cancer as described herein include, without limitation, cytokine therapy, immune checkpoint inhibitors, and the like. Examples of immunotherapies that can be used to treat a patient (e.g., a human patient) identified as having pancreatic cancer as described herein include, without limitation, chimeric antigen receptor (CAR) T-cell immunotherapy, and the like.

### 5.3. Method for Determining THBS2 Cutoff Value

In certain embodiments, the presently disclosed subject matter provides a method for determining a cutoff value of THBS2 for diagnosis of pancreatic cancer in a subject, comprising: measuring the distribution of THBS2 values in a group of healthy subjects; selecting a THBS2 value wherein the selected THBS2 value has a false positive rate of between about 0 to about 5 percent; and measuring the sensitivity value and specificity value of the selected THBS2 value in detecting pancreatic cancer in a group of subjects having pancreatic cancer, wherein the sensitivity value of at least about 50% and the specificity value of at least about 90% indicate the selected THBS2 value is a cutoff value of THBS2 for diagnosis of pancreatic cancer in a subject.

In certain embodiments, the healthy subjects are healthy controls of a clinical study. In certain embodiments, the healthy subjects are combined healthy controls from a number of clinical study.

In certain embodiments, the selected THBS2 value has a false positive rate of between about 0 to about 5%, between about 0 to about 0.05%, between about 0 to about 0.1%, between about 0 to about 0.5%, between about 0.5 to about 1%, between about 1 to about 2%, between about 2 to about 3%, between about 3 to about 4%, or between about 4 to about 5%. In certain embodiments, the selected THBS2 value has a false positive rate of about 0%. In certain embodiments, the selected THBS2 value has a false positive rate of about 1%. In certain embodiments, the selected THBS2 value has a false positive rate of about 2%. In certain embodiments, the selected THBS2 value has a false positive rate of about 3%. In certain embodiments, the selected THBS2 value has a false positive rate of about 4%. In certain embodiments, the selected THBS2 value has a false positive rate of about 5%.

In certain embodiments, the sensitivity value of the selected THBS2 value is between about 40% to 100% and the specificity value of the selected THBS2 value is between about 90% to 100% to indicate for diagnosis of pancreatic cancer in a subject. In certain embodiments, the sensitivity value is at least about 40%, at least about 45%, at least about 50%, at least about 55%, at least about 58%, at least about 60%. In certain embodiments, the sensitivity value is about 50%. In certain embodiments, the sensitivity value is about 51%. In certain embodiments, the sensitivity value is about 53%. In certain embodiments, the sensitivity value is about 57%. In certain embodiments, the sensitivity value is about 58%. In certain embodiments, the specificity value is at least about 90%, at least about 91%, at least about 92%, at least about 93%, at least about 94%, at least about 95%, at least about 96%, at least about 97%, at least about 98%, at least about 99%. In certain embodiments, the specificity value is about 99%. In certain embodiments, the specificity value is about 97%. In certain embodiments, the specificity value is about 100%.

### 5.4. Biomarker Detection

A biomarker used in the methods of the disclosure (e.g., THBS2, or THBS2/CA19-9 panel) can be identified in a biological sample using any method known in the art. Determining the presence of a biomarker, protein or degradation product thereof, the presence of mRNA or pre-mRNA, or the presence of any biological molecule or product that is indicative of biomarker expression, or degradation product thereof, can be carried out for use in the methods of the disclosure by any method described herein or known in the art.

Protein Detection Techniques

Methods for the detection of protein biomarkers are well known to those skilled in the art, and include but are not limited to mass spectrometry techniques, 1-D or 2-D gel-based analysis systems, chromatography, enzyme linked immunosorbent assays (ELISA), radioimmunoassays (RIA), enzyme immunoassays (EIA), Western Blotting, immunoprecipitation and immunohistochemistry. These methods use antibodies, or antibody equivalents, to detect protein, or use biophysical techniques. Antibody arrays or protein chips can also be employed, see for example U.S. Patent Application Nos: 2003/0013208A1; 2002/0155493A1, 2003/0017515 and U.S. Pat. Nos. 6,329,209 and 6,365,418, herein incorporated by reference in their entireties.

ELISA and RIA procedures can be conducted such that a biomarker standard is labeled (with a radioisotope such as ¹²⁵I or ³⁵S, or an assayable enzyme, such as horseradish peroxidase or alkaline phosphatase), and, together with the unlabeled sample, brought into contact with the corresponding antibody, whereon a second antibody is used to bind the first, and radioactivity or the immobilized enzyme assayed (competitive assay). Alternatively, the biomarker in the sample is allowed to react with the corresponding immobilized antibody, radioisotope or enzyme-labeled anti-biomarker antibody is allowed to react with the system, and radioactivity or the enzyme assayed (ELISA-sandwich assay). Other conventional methods can also be employed as suitable.

The above techniques can be conducted essentially as a “one-step” or “two-step” assay. A “one-step” assay involves contacting antigen with immobilized antibody and, without washing, contacting the mixture with labeled antibody. A “two-step” assay involves washing before contacting the mixture with labeled antibody. Other conventional methods can also be employed as suitable.

In certain embodiments, a method for measuring biomarker expression includes the steps of: contacting a biological sample, e.g., blood and/or plasma, with an antibody or variant (e.g., fragment) thereof which selectively binds the biomarker, and detecting whether the antibody or variant thereof is bound to the sample. A method can further include contacting the sample with a second antibody, e.g., a labeled antibody. The method can further include one or more steps of washing, e.g., to remove one or more reagents.

It can be desirable to immobilize one component of the assay system on a support, thereby allowing other components of the system to be brought into contact with the component and readily removed without laborious and time-consuming labor. It is possible for a second phase to be immobilized away from the first, but one phase is usually sufficient.

It is possible to immobilize the enzyme itself on a support, but if solid-phase enzyme is required, then this is generally best achieved by binding to antibody and affixing the antibody to a support, models and systems for which are well-known in the art. Simple polyethylene can provide a suitable support.

Enzymes employable for labeling are not particularly limited, but can be selected, for example, from the members of the oxidase group. These catalyze production of hydrogen peroxide by reaction with their substrates, and glucose oxidase is often used for its good stability, ease of availability and cheapness, as well as the ready availability of its substrate (glucose). Activity of the oxidase can be assayed by measuring the concentration of hydrogen peroxide formed after reaction of the enzyme-labeled antibody with the substrate under controlled conditions well-known in the art.

Other techniques can be used to detect a biomarker according to a practitioner's preference based upon the present disclosure. One such technique that can be used for detecting and quantitating biomarker protein levels is Western blotting (Towbin et al., Proc. Nat. Acad. Sci. 76:4350 (1979)). Cells can be frozen, homogenized in lysis buffer, and the lysates subjected to SDS-PAGE and blotting to a membrane, such as a nitrocellulose filter. Antibodies (unlabeled) are then brought into contact with the membrane and assayed by a secondary immunological reagent, such as labeled protein A or anti-immunoglobulin (suitable labels including ¹²⁵I, horseradish peroxidase and alkaline phosphatase). Chromatographic detection can also be used. In certain embodiments, immunodetection can be performed with antibody to a biomarker using the enhanced chemiluminescence system (e.g., from PerkinElmer Life Sciences, Boston, Mass.). The membrane can then be stripped and re-blotted with a control antibody, e.g., anti-actin (A-2066) polyclonal antibody from Sigma (St. Louis, Mo.).

Immunohistochemistry can be used to detect the expression and/ presence of a biomarker, e.g., in a biopsy sample. A suitable antibody is brought into contact with, for example, a thin layer of cells, followed by washing to remove unbound antibody, and then contacted with a second, labeled, antibody. Labeling can be by fluorescent markers, enzymes, such as peroxidase, avidin or radiolabeling. The assay is scored visually, using microscopy and the results can be quantitated.

Other machine or autoimaging systems can also be used to measure immunostaining results for the biomarker. As used herein, “quantitative” immunohistochemistry refers to an automated method of scanning and scoring samples that have undergone immunohistochemistry, to identify and quantitate the presence of a specified biomarker, such as an antigen or other protein. The score given to the sample is a numerical representation of the intensity of the immunohistochemical staining of the sample, and represents the amount of target biomarker present in the sample. As used herein, Optical Density (OD) is a numerical score that represents intensity of staining. As used herein, semi-quantitative immunohistochemistry refers to scoring of immunohistochemical results by human eye, where a trained operator ranks results numerically (e.g., as 1, 2 or 3).

Various automated sample processing, scanning and analysis systems suitable for use with immunohistochemistry are available in the art. Such systems can include automated staining (see, e.g., the Benchmark system, Ventana Medical Systems, Inc.) and microscopic scanning, computerized image analysis, serial section comparison (to control for variation in the orientation and size of a sample), digital report generation, and archiving and tracking of samples (such as slides on which tissue sections are placed). Cellular imaging systems are commercially available that combine conventional light microscopes with digital image processing systems to perform quantitative analysis on cells and tissues, including immunostained samples. See, e.g., the CAS-200 system (Becton, Dickinson & Co.).

Antibodies against biomarkers can also be used for imaging purposes, for example, to detect the presence of a biomarker in cells of a subject. Suitable labels include radioisotopes, iodine (¹²⁵I, ¹²¹I) carbon (¹⁴C), sulphur (³⁵S), tritium (³H), indium (¹¹²In)^{,} and technetium (^{99m}Tc), fluorescent labels, such as fluorescein and rhodamine and biotin. Immunoenzymatic interactions can be visualized using different enzymes such as peroxidase, alkaline phosphatase, or different chromogens such as DAB, AEC or Fast Red.

For in vivo imaging purposes, antibodies are not detectable, as such, from outside the body, and so must be labeled, or otherwise modified, to permit detection. Markers for this purpose can be any that do not substantially interfere with the antibody binding, but which allow external detection. Suitable markers can include those that can be detected by X-radiography, NMR or MRI. For X-radiographic techniques, suitable markers include any radioisotope that emits detectable radiation but that is not overtly harmful to the subject, such as barium or caesium, for example. Suitable markers for NMR and MRI generally include those with a detectable characteristic spin, such as deuterium, which can be incorporated into the antibody by suitable labeling of nutrients for the relevant hybridoma, for example.

The size of the subject, and the imaging system used, will determine the quantity of imaging moiety needed to produce diagnostic images. In the case of a radioisotope moiety, for a human subject, the quantity of radioactivity injected will normally range from about 5 to 20 millicuries of technetium-99m.

The labeled antibody or antibody fragment will then preferentially accumulate at the location of cells which contain a biomarker. The labeled antibody or variant thereof, e.g., antibody fragment, can then be detected using known techniques. Antibodies include any antibody, whether natural or synthetic, full length or a fragment thereof, monoclonal or polyclonal, that binds sufficiently strongly and specifically to the biomarker to be detected. An antibody can have a K_{d} of at most about 10⁻⁶M, 10⁻⁷M, 10⁻⁸M, 10⁻⁹M, 10⁻¹⁰M, 10¹¹M, 10¹²M. The phrase “specifically binds” refers to binding of, for example, an antibody to an epitope or antigen or antigenic determinant in such a manner that binding can be displaced or competed with a second preparation of identical or similar epitope, antigen or antigenic determinant.

Antibodies and derivatives thereof that can be used encompasses polyclonal or monoclonal antibodies, chimeric, human, humanized, primatized (CDR-grafted), veneered or single-chain antibodies, phase produced antibodies (e.g., from phage display libraries), as well as functional binding fragments, of antibodies. For example, antibody fragments capable of binding to a biomarker, or portions thereof, including, but not limited to Fv, Fab, Fab′ and F(ab′)₂ fragments can be used. Such fragments can be produced by enzymatic cleavage or by recombinant techniques. For example, papain or pepsin cleavage can generate Fab or F(ab′)₂ fragments, respectively. Other proteases with the requisite substrate specificity can also be used to generate Fab or F(ab′)₂ fragments. Antibodies can also be produced in a variety of truncated forms using antibody genes in which one or more stop codons have been introduced upstream of the natural stop site. For example, a chimeric gene encoding a F(ab′)₂ heavy chain portion can be designed to include DNA sequences encoding the CH, domain and hinge region of the heavy chain.

Synthetic and engineered antibodies are described in, e.g., Cabilly et al., U.S. Pat. No. 4,816,567 Cabilly et al., European Patent No. 0,125,023 B1; Boss et al., U.S. Pat. No. 4,816,397; Boss et al., European Patent No. 0,120,694 B1; Neuberger, M. S. et al., WO 86/01533; Neuberger, M. S. et al., European Patent No. 0,194,276 B1; Winter, U.S. Pat. No. 5,225,539; Winter, European Patent No. 0,239,400 B1; Queen et al., European Patent No. 0451216 B1; and Padlan, E. A. et al., EP 0519596 A1. See also, Newman, R. et al., BioTechnology, 10: 1455-1460 (1992), regarding primatized antibody, and Ladner et al., U.S. Pat. No. 4,946,778 and Bird, R. E. et al., Science, 242: 423-426 (1988)) regarding single-chain antibodies.

In certain embodiments, agents that specifically bind to a polypeptide other than antibodies are used, such as peptides. Peptides that specifically bind can be identified by any means known in the art, e.g., peptide phage display libraries. Generally, an agent that is capable of detecting a biomarker polypeptide, such that the presence of a biomarker is detected and/or quantitated, can be used. As defined herein, an “agent” refers to a substance that is capable of identifying or detecting a biomarker in a biological sample (e.g., identifies or detects the mRNA of a biomarker, the DNA of a biomarker, the protein of a biomarker). In certain embodiments, the agent is a labeled or labelable antibody which specifically binds to a biomarker polypeptide.

In addition, a biomarker can be detected using Mass Spectrometry such as MALDI/TOF (time-of-flight), SELDI/TOF, liquid chromatography-mass spectrometry (LC-MS), gas chromatography-mass spectrometry (GC-MS), high performance liquid chromatography-mass spectrometry (HPLC-MS), capillary electrophoresis-mass spectrometry, nuclear magnetic resonance spectrometry, or tandem mass spectrometry (e.g., MS/MS, MS/MS/MS, ESI-MS/MS, etc.). See for example, U.S. Patent Application Nos: 20030199001, 20030134304, 20030077616, which are herein incorporated by reference.

Mass spectrometry methods are well known in the art and have been used to quantify and/or identify biomolecules, such as proteins (see, e.g., Li et al. (2000) Tibtech 18:151-160; Rowley et al. (2000) Methods 20: 383-397; and Kuster and Mann (1998) Curr. Opin. Structural Biol. 8: 393-400). Further, mass spectrometric techniques have been developed that permit at least partial de novo sequencing of isolated proteins. Chait et al., Science 262:89-92 (1993); Keough et al., Proc. Natl. Acad. Sci. USA. 96:7131-6 (1999); reviewed in Bergman, EXS 88:133-44 (2000).

In certain embodiments, a gas phase ion spectrophotometer is used. In other embodiments, laser-desorption/ionization mass spectrometry is used to analyze the sample. Modem laser desorption/ionization mass spectrometry (“LDI-MS”) can be practiced in two main variations: matrix assisted laser desorption/ionization (“MALDI”) mass spectrometry and surface-enhanced laser desorption/ionization (“SELDI”). In MALDI, the analyte is mixed with a solution containing a matrix, and a drop of the liquid is placed on the surface of a substrate. The matrix solution then co-crystallizes with the biological molecules. The substrate is inserted into the mass spectrometer. Laser energy is directed to the substrate surface where it desorbs and ionizes the biological molecules without significantly fragmenting them. However, MALDI has limitations as an analytical tool. It does not provide means for fractionating the sample, and the matrix material can interfere with detection, especially for low molecular weight analytes. See, e.g., U .S. Pat. No. 5,118,937 (Hillenkamp et al.), and U.S. Pat. No. 5,045,694 (Beavis & Chait).

For additional information regarding mass spectrometers, see, e.g., Principles of Instrumental Analysis, 3rd edition. Skoog, Saunders College Publishing, Philadelphia, 1985; and Kirk-Othmer Encyclopedia of Chemical Technology, 4th ed. Vol. 15 (John Wiley & Sons, New York 1995), pp. 1071-1094.

Detection of the presence of a marker or other substances will typically involve detection of signal intensity. This, in turn, can reflect the quantity and character of a polypeptide bound to the substrate. For example, in certain embodiments, the signal strength of peak values from spectra of a first sample and a second sample can be compared (e.g., visually, by computer analysis etc.), to determine the relative amounts of a particular biomarker. Software programs such as the Biomarker Wizard program (Ciphergen Biosystems, Inc., Fremont, Calif.) can be used to aid in analyzing mass spectra. The mass spectrometers and their techniques are well known to those of skill in the art.

Any person skilled in the art understands, any of the components of a mass spectrometer (e.g., desorption source, mass analyzer, detect, etc.) and varied sample preparations can be combined with other suitable components or preparations described herein, or to those known in the art. For example, in certain embodiments a control sample can contain heavy atoms (e.g., ¹³C) thereby permitting the test sample to be mixed with the known control sample in the same mass spectrometry run.

In certain embodiments, a laser desorption time-of-flight (TOF) mass spectrometer is used. In laser desorption mass spectrometry, a substrate with a bound marker is introduced into an inlet system. The marker is desorbed and ionized into the gas phase by laser from the ionization source. The ions generated are collected by an ion optic assembly, and then in a time-of-flight mass analyzer, ions are accelerated through a short high voltage field and let drift into a high vacuum chamber. At the far end of the high vacuum chamber, the accelerated ions strike a sensitive detector surface at a different time. Since the time-of-flight is a function of the mass of the ions, the elapsed time between ion formation and ion detector impact can be used to identify the presence or absence of molecules of specific mass to charge ratio.

In certain embodiments, the relative amounts of one or more biomarkers present in a first or second sample is determined, in part, by executing an algorithm with a programmable digital computer. The algorithm identifies at least one peak value in the first mass spectrum and the second mass spectrum. The algorithm then compares the signal strength of the peak value of the first mass spectrum to the signal strength of the peak value of the second mass spectrum of the mass spectrum. The relative signal strengths are an indication of the amount of the biomarker that is present in the first and second samples. A standard containing a known amount of a biomarker can be analyzed as the second sample to better quantify the amount of the biomarker present in the first sample. In certain embodiments, the identity of the biomarkers in the first and second sample can also be determined.

RNA Detection Techniques

Any method for qualitatively or quantitatively detecting a nucleic acid biomarker can be used. Detection of RNA transcripts can be achieved, for example, by Northern blotting, wherein a preparation of RNA is run on a denaturing agarose gel, and transferred to a suitable support, such as activated cellulose, nitrocellulose or glass or nylon membranes. Radiolabeled cDNA or RNA is then hybridized to the preparation, washed and analyzed by autoradiography.

Detection of RNA transcripts can further be accomplished using amplification methods. For example, it is within the scope of the present disclosure to reverse transcribe mRNA into cDNA followed by polymerase chain reaction (RT-PCR); or, to use a single enzyme for both steps as described in U.S. Pat. No. 5,322,770, or reverse transcribe mRNA into cDNA followed by symmetric gap ligase chain reaction (RT-AGLCR) as described by R. L. Marshall, et al., PCR Methods and Applications 4: 80-84 (1994).

In certain embodiments, quantitative real-time polymerase chain reaction (qRT-PCR) is used to evaluate mRNA levels of biomarker. The levels of a biomarker and a control mRNA can be quantitated in cancer tissue or cells and adjacent benign tissues. In one specific embodiment, the levels of one or more biomarkers can be quantitated in a biological sample.

Other known amplification methods which can be utilized herein include but are not limited to the so-called “NASBA” or “3SR” technique described in PNAS USA 87: 1874-1878 (1990) and also described in Nature 350 (No. 6313): 91-92 (1991); Q-beta amplification as described in published European Patent Application (EPA) No. 4544610; strand displacement amplification (as described in G. T. Walker et al., Clin. Chem. 42: 9-13 (1996) and European Patent Application No. 684315; and target mediated amplification, as described by PCT Publication W09322461.

In situ hybridization visualization can also be employed, wherein a radioactively labeled antisense RNA probe is hybridized with a thin section of a biopsy sample, washed, cleaved with RNase and exposed to a sensitive emulsion for autoradiography. The samples can be stained with haematoxylin to demonstrate the histological composition of the sample, and dark field imaging with a suitable light filter shows the developed emulsion. Non-radioactive labels such as digoxigenin can also be used.

Another method for evaluation of biomarker expression is to detect mRNA levels of a biomarker by fluorescent in situ hybridization (FISH). FISH is a technique that can directly identify a specific region of DNA or RNA in a cell and therefore enables to visual determination of the biomarker expression in tissue samples. The FISH method has the advantages of a more objective scoring system and the presence of a built-in internal control consisting of the biomarker gene signals present in all non-neoplastic cells in the same sample. Fluorescence in situ hybridization is a direct in situ technique that is relatively rapid and sensitive. FISH test also can be automated. Immunohistochemistry can be combined with a FISH method when the expression level of the biomarker is difficult to determine by immunohistochemistry alone.

Alternatively, mRNA expression can be detected on a DNA array, chip or a microarray. Oligonucleotides corresponding to the biomarker(s) are immobilized on a chip which is then hybridized with labeled nucleic acids of a test sample obtained from a subject. Positive hybridization signal is obtained with the sample containing biomarker transcripts. Methods of preparing DNA arrays and their use are well known in the art. (See, for example, U.S. Pat. Nos. 6,618,6796; 6,379,897; 6,664,377; 6,451,536; 548,257; U.S. 20030157485 and Schena et al. 1995 Science 20:467-470; Gerhold et al. 1999 Trends in Biochem. Sci. 24, 168-173; and Lennon et al. 2000 Drug discovery Today 5: 59-65, which are herein incorporated by reference in their entirety). Serial Analysis of Gene Expression (SAGE) can also be performed (See for example U.S. Patent Application 20030215858).

To monitor mRNA levels, for example, mRNA can be extracted from the biological sample to be tested, reverse transcribed and fluorescent-labeled cDNA probes are generated. The microarrays capable of hybridizing to a biomarker, cDNA can then probed with the labeled cDNA probes, the slides scanned and fluorescence intensity measured. This intensity correlates with the hybridization intensity and expression levels.

Types of probes for detection of RNA include cDNA, riboprobes, synthetic oligonucleotides and genomic probes. The type of probe used will generally be dictated by the particular situation, such as riboprobes for in situ hybridization, and cDNA for Northern blotting, for example. In certain embodiments, the probe is directed to nucleotide regions unique to the particular biomarker RNA. The probes can be as short as is required to differentially recognize the particular biomarker mRNA transcripts, and can be as short as, for example, 15 bases; however, probes of at least 17 bases, at least 18 bases and at least 20 bases can be used. In certain embodiments, the primers and probes hybridize specifically under stringent conditions to a nucleic acid fragment having the nucleotide sequence corresponding to the target gene. As herein used, the term “stringent conditions” means hybridization will occur only if there is at least 95% or at least 97% identity between the sequences.

The form of labeling of the probes can be any that is appropriate, such as the use of radioisotopes, for example, ³²P and ³⁵5. Labeling with radioisotopes can be achieved, whether the probe is synthesized chemically or biologically, by the use of suitably labeled bases.

### 5.5. Kit

In certain embodiments, the presently disclosed subject matter provides for a kit for diagnosing whether a subject has pancreatic cancer, comprising reagents useful for detecting THBS2 in one or biological samples from the subject. The presently disclosed subject matter also provides for a kit for diagnosing whether a subject has pancreatic cancer, comprising reagents useful for detecting THBS2 and CA19-9 in one or biological samples from the subject. In certain embodiments, when the subject is diagnosed with pancreatic cancer, an anti-cancer treatment is recommended for the subject.

Types of kits include, but are not limited to, packaged probe and primer sets (e.g., TaqMan probe/primer sets), arrays/microarrays, biomarker-specific antibodies and beads, which further contain one or more probes, primers or other detection reagents for detecting one or more biomarkers of the present disclosure.

In certain embodiments, a kit can include a pair of oligonucleotide primers suitable for polymerase chain reaction (PCR) or nucleic acid sequencing, for detecting one or more biomarker(s) to be identified (e.g., THBS2 or a panel of THBS2 and CA19-9). A pair of primers can include nucleotide sequences complementary to the biomarker (e.g., THBS2 or a panel of THBS2 and CA19-9), and can be of sufficient length to selectively hybridize with said biomarker. Alternatively, the complementary nucleotides can selectively hybridize to a specific region in close enough proximity 5′ and/or 3′ to the biomarker position to perform PCR and/or sequencing. Multiple biomarker-specific primers can be included in the kit to simultaneously assay large number of biomarkers. The kit can also include one or more polymerases, reverse transcriptase and nucleotide bases, wherein the nucleotide bases can be further detectably labeled.

In certain embodiments, a primer can be at least about 10 nucleotides or at least about 15 nucleotides or at least about 20 nucleotides in length and/or up to about 200 nucleotides or up to about 150 nucleotides or up to about 100 nucleotides or up to about 75 nucleotides or up to about 50 nucleotides in length.

In certain embodiments, the oligonucleotide primers can be immobilized on a solid surface or support, for example, on a nucleic acid microarray, wherein the position of each oligonucleotide primer bound to the solid surface or support is known and identifiable.

In a certain, non-limiting embodiment, a kit can include at least one nucleic acid probe, suitable for in situ hybridization or fluorescent in situ hybridization, for detecting the biomarker(s) to be identified. Such kits will generally include one or more oligonucleotide probes that have specificity for various biomarkers.

In certain non-limiting embodiments, a kit can include a primer for detection of a biomarker by primer extension.

In certain non-limiting embodiments, a kit can include at least one antibody for immunodetection of the biomarker(s) to be identified (e.g., THBS2 or a panel of THBS2 and CA19-9). Antibodies, both polyclonal and monoclonal, specific for a biomarker, can be prepared using conventional immunization techniques, as will be generally known to those of skill in the art. The immunodetection reagents of the kit can include detectable labels that are associated with, or linked to, the given antibody or antigen itself. Such detectable labels include, for example, chemiluminescent or fluorescent molecules (rhodamine, fluorescein, green fluorescent protein, luciferase, Cy3, Cy5 or ROX), radiolabels (³H, ³⁵S, ³²P, ¹⁴C, ¹³¹I) or enzymes (alkaline phosphatase, horseradish peroxidase).

In a certain non-limiting embodiment, the biomarker-specific antibody (e.g., antibody for THBS2 or CA19-9) can be provided bound to a solid support, such as a column matrix, an array, or well of a microtiter plate. Alternatively, the support can be provided as a separate element of the kit.

In certain non-limiting embodiments, a kit can include one or more primers, probes, microarrays, or antibodies suitable for detecting a panel of THBS2 and CA19-9 biomarkers.

In certain non-limiting embodiments, a biomarker detection kit can include one or more detection reagents and other components (e.g., a buffer, enzymes such as DNA polymerases or ligases, chain extension nucleotides such as deoxynucleotide triphosphates, and in the case of Sanger-type DNA sequencing reactions, chain terminating nucleotides, positive control sequences, negative control sequences, and the like) necessary to carry out an assay or reaction to detect a biomarker. A kit can also include additional components or reagents necessary for the detection of a biomarker, such as secondary antibodies for use in western blotting immunohistochemistry. A kit can further include one or more other biomarkers or reagents for evaluating other prognostic factors, e.g., tumor stage.

A kit can further contain means for comparing the biomarker with a cutoff value, and can include instructions for using the kit to detect the biomarker of interest. For example, the instructions can describe that an increase in the level of the THBS2 as compared to a THBS2 cutoff value indicates that the subject has pancreatic cancer. In certain embodiments, the kit further comprises an instruction describing that an increase in the level of the THBS2 as compared to a THBS2 cut-off value and/or an increase in the level of the CA19-9 as compared to a CA19-9 cutoff value indicates that the subject has pancreatic cancer.

In certain embodiments the CA19-9 cutoff value is a CA19-9 protein cutoff value. In certain embodiments, the CA19-9 protein cutoff value is between about 40 to 45 U/ml, between about 45 to 55 U/ml, between about 50 to 55 U/ml, or between about 55 to 60 U/ml. In certain embodiments, the CA19-9 protein cutoff value is about 55 U/ml.

In certain embodiments the THBS2 cutoff value is a THBS2 protein cutoff value. In certain embodiments, the THBS2 cutoff value is between about 20 to about 100 ng/ml, between about 20 to about 25 ng/ml, between about 25 to about 30 ng/ml, between about 30 to about 35 ng/ml, between about 35 to about 40 ng/ml between about 40 to about 45 ng/ml, between about 45 to about 50 ng/ml, between about 50 to about 55 ng/ml between about 55 to about 60 ng/ml, between about 60 to about 65 ng/ml between about 65 to about 70 ng/ml, between about 70 to about 75 ng/ml, between about 75 to about 80 ng/ml, between about 85 to about 90 ng/ml, between about 90 to about 95 ng/ml, or between about 95 to about 100 ng/ml. In certain embodiments, the THBS2 cutoff value is about 36 ng/ml, about 37 ng/ml, about 38 ng/ml, about 39 ng/ml, about 40 ng/ml, about 41 ng/ml, about 42 ng/ml, about 43 ng/ml, about 44 ng/ml, or about 45 ng/ml. In certain embodiments, the THBS2 cutoff value is about 42 ng/ml.

### EXAMPLES

The following Examples are offered to more fully illustrate the disclosure, but are not to be construed as limiting the scope thereof

### Example 1

### Combined THBS2 and CA19-9 Blood Based Markers Detect Early Pancreatic Ductal Adenocarcinoma

Introduction

Biomarkers have been sought to facilitate early detection of pancreatic ductal adenocarcinoma (PDAC), which is often diagnosed too late for effective therapy.

Starting with a PDAC cell reprogramming model that recapitulates human PDAC progression, from which secreted and released proteins have been identified, a subset of proteins as potential plasma biomarkers of PDAC have been tested and validated. The proteins released from precursor lesions, such as pancreatic intraepithelial neoplasia (e.g., PanIN2 and PanIN3) (24) progressing to PDAC, might provide an innovative and effective opportunity for discovering diagnostic biomarkers. Recurrent, advanced human PDAC cells were previously reprogrammed into an induced pluripotent stem (iPS) cell-like line (25). The iPS-like line (designated as 10-22 cells), can be propagated indefinitely, yet preferentially generates PanIN2/3 ductal lesions after growing for 3 months as teratomas in immunodeficient mice. The lesions progress to invasive PDAC by 6-9 months. Proteomic analysis of conditioned medium from 10-22 cell-derived PaniNs cultured as organoids, in comparison to control conditions, revealed 107 human proteins specific to the PanIN2/3 secreted or released proteome (25). Of these, 43 proteins fell into interconnected TGFβ and integrin networks for PDAC progression (26, 27) and 25 proteins were within a network for the transcription factor HNF4a, which it was discovered to be dynamic in PDAC progression (25).

The instant study reports an analysis of proteins , secreted or released from the 10-22 cell-derived PanIN organoids, as a PDAC biomarker panel in human plasma samples from a single institution, using a phased cancer biomarker development design that incorporated criteria for prospective specimen collection, retrospective blinded evaluation (PRoBE) (28, 29).

Results

ELISA was optimized with independent investigations of plasma samples from patients with various stages of PDAC, from individuals with benign pancreatic disease, and from healthy controls. Phase 1 discovery (N=20), Phase 2a validation (N=189), and Phase 2b validation (N=537) studies were designed using recommended rigorous criteria for early detection biomarker development. It was found that plasma Thrombospondin-2 (THBS2) concentrations discriminated all stages of PDAC consistently over the three investigations, with a Receiver Operating Characteristic (ROC) c-statistic=0.76 in Phase 1, 0.842 in Phase 2a, and 0.875 in Phase 2b, performing as well in resectable Stage I cancer as in Stage III/IV cancer. THBS2 concentrations combined with those for CA19-9, a previously identified PDAC biomarker, yielded c-statistics of 0.956 in the Phase 2a study and 0.970 in the Phase 2b study. THBS2 data improved the ability of CA19-9 to distinguish PDAC from pancreatitis. With a specificity of 98%, THBS2 and CA19-9 combined yield a sensitivity of 87% for PDAC in the larger Phase 2b study. Given this, a THBS2 and CA19-9 panel assessed in human blood using conventional ELISA assays improve the detection of high risk patients with PDAC.

Discovery studies: Of the 107 proteins secreted and released selectively by human PanIN organoids (25), 53 proteins were focused on for their low abundance (<2 nmol) in the healthy human plasma proteome and RNA-Seq databases (30-32) (Table 3). Enzyme-linked immunosorbent assays (ELISAs) from validated sources (33) were not available for most of these rarely expressed proteins. Of the proteins for which reliable ELISA kits were available and were not implicated as markers in other diseases, it was focused on MMP2, MMP10, and Thrombospondin-2 (THBS2) because they occur in integrated networks for TGF-f3 and integrin signaling that drives PDAC (25). The three candidates in a screen of human plasma samples were therefore investigated. All procedures were performed using a recommended biomarker phased design following the PRoBE criteria (28, 29). De-identified human plasma samples from the Mayo Clinic pancreas research biospecimen repository were shipped to the lab, which performed ELISA analyses blinded to disease status, and then returned coded data to the Mayo Clinic team for statistical analysis and interpretation.

TABLE 3
List of 53 proteins secreted or released from 10-22 cell, PanIN-stage lesions that
are at low abundance in healthy human plasma proteome and RNA-Seq databases.

| | | PDAC Network | Identified | Identified in | | |
|---|---|---|---|---|---|---|
| | | from Kim | in plasma | plasma MOPED | | |
| | | et al. 2013 | protein DB | database As | | ELISA kits |
| IPI number | Protein | Cell Reports | (as of 2014) | of 2014 | | available |
| IPI00152881 | SHROOM3 | TGFb/Integrin | yes | 0.1 | nmol | no |
| IPI00215893 | HMOX1 | TGFb/Integrin | yes | 0 | nmol | no |
| IPI00007960 | *PERIOSTIN | TGFb/Integrin | yes | no information | | yes |
| IPI00013405 | MMP10 | TGF/Integrin | no | 0 | nmol | yes |
| IPI00027780 | MMP-2 | TGF/Integrin | no | 2 | nmol | yes |
| IPI00018769 | THBS2 | TGF/Integrin | yes | no information | | yes |
| IPI00009841 | EWSR1 | TGFb/Integrin | yes | no information | | no |
| IPI00005776 | NOD1 | TGF/Integrin | yes | 1 | nmol | no |
| IPI00220986 | ADAMTS9 | TGF/Integrin | yes | 1 | nmol | no |
| IPI00022443 | *AFP | TGF/Integrin | yes | 1 | nmol | yes |
| IPI00247295 | SYNE1 | TGF/Integrin | yes | 0.2 | nmol | no |
| IPI00239405 | SYNE2 | TGFb/Integrin | yes | no information | | no |
| IPI00008315 | EPHB1 | TGFb/Integrin | yes | 0 | nmol | no |
| IPI00218292 | UFD1L | TGFb/Integrin | no | 0 | nmol | no |
| IPI00002901 | TEAD1 | TGFb/Integrin | no | 0 | nmol | no |
| IPI00027280 | TOP2B | TGFb/Integrin | yes | 0.1-1 | nmol | no |
| IPI00217185 | RYR3 | TGF/Integrin | yes | 0.8 | nmol | no |
| IPI00166612 | CMYA5 | TGF/Integrin | yes | 0 | nmol | no |
| IPI00221255 | MYLK | TGF/Integrin | yes | 0.2 | nmol | no |
| IPI00251161 | KIAA1109 | RAS/p53/JUN/ | yes | 0.1 | nmol | no |
| | | CTNB1 | | | | |
| IPI00398020 | ODZ3 | RAS/p53/JUN/ | yes | no information | | no |
| | | CTNB1 | | | | |
| IPI00235481 | PMFBP1 | RAS/p53/JUN/ | yes | 0.1 | nmol | no |
| | | CTNB1 | | | | |
| IPI00289329 | EPHB3 | RAS/p53/JUN/ | yes | no information | | no |
| | | CTNB1 | | | | |
| IPI00177498 | LIMCH1 | RAS/p53/JUN/ | yes | no information | | no |
| | | CTNB1 | | | | |
| IPI00159322 | TCF20 | RAS/p53/JUN/ | yes | 0.1 | nmol | no |
| | | CTNB1 | | | | |
| IPI00847609 | SVEP1 | no network | yes | 0.1 | nmol | no |
| IPI00396634 | KIAA1671 | no network | no | 0 | nmol | no |
| IPI00292836 | KIAA1529 | no network | yes | 0 | nmol | no |
| IPI00896378 | GNN | no network | no | no information | | no |
| IPI00420019 | DOS | no network | no | 0.5 | nmol | no |
| IPI00293887 | STARD8 | no network | yes | 0 | nmol | no |
| | (DLC3) | | | | | |
| IPI00183041 | SCN8A | no network | yes | 0.5 | nmol | no |
| IPI00143753 | U2SURP | no network | no | 0 | nmol | no |
| IPI00031104 | TCHP | no network | yes | 1 | nmol | no |
| IPI00012829 | RAD51C | no network | yes | no information | | no |
| IPI00024804 | ATP2A1 | no network | yes | 0 | nmol | no |
| IPI00377214 | NLRX1 | no network | no | 0 | nmol | no |
| IPI00028833 | ZNF160 | no network | yes | 1 | nmol | no |
| IPI00645947 | RTTN | no network | no | 0 | nmol | no |
| IPI00328762 | ABCA13 | no network | yes | 0 | nmol | no |
| IPI00288940 | OBSCN | HNF4a | yes | 0.1 | nmol | no |
| IPI00011385 | LOXL3 | HNF4a | yes | 0 | nmol | no |
| IPI00029046 | MLEC | HNF4a | yes | 0 | nmol | no |
| IPI00002127 | DNAH1 | HNF4a | yes | 0 | nmol | no |
| IPI00152653 | DNAH5 | HNF4a | yes | 1 | nmol | no |
| IPI00412106 | DNAH12 | HNF4a | yes | 1 | nmol | no |
| IPI00888430 | DNAH17 | HNF4a | no | 0 | nmol | no |
| IPI00396218 | SCYL2 | HNF4a | yes | 0 | nmol | no |
| IPI00303300 | FKBP10 | HNF4a | no | 0 | nmol | no |
| IPI00002320 | FLRT3 | HNF4a | yes | 0 | nmol | no |
| IPI00007256 | ZHX2(AFR1) | HNF4a | yes | 0 | nmol | no |
| IPI00375560 | ZNF804A | HNF4a | no | 0.1 | nmol | no |
| IPI00019884 | ACTN2 | HNF4a | yes | 0.1 | nmol | no |

*AFP and PERIOSTIN were not chosen because of their indication in hepatocellular carcinoma and breast cancer, respectively.

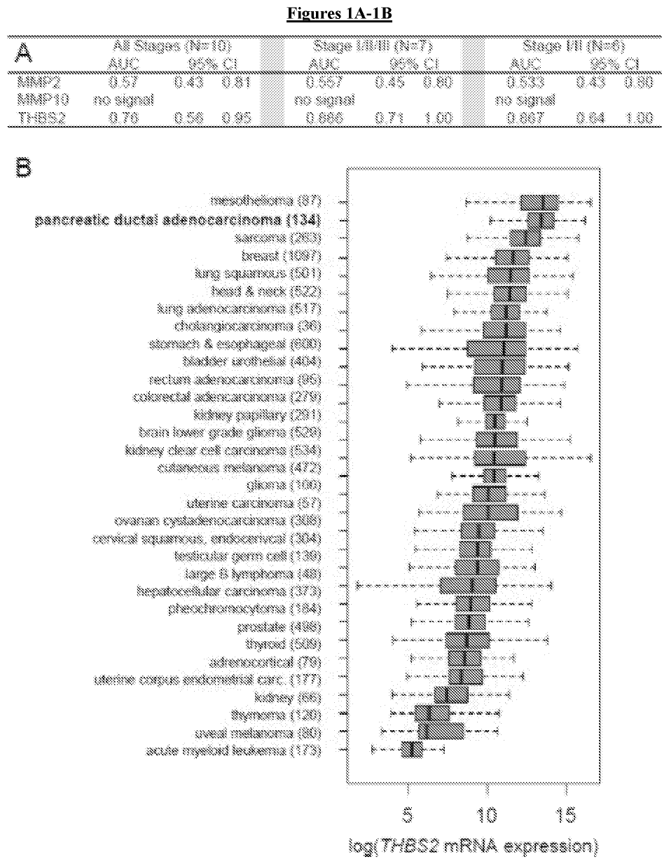

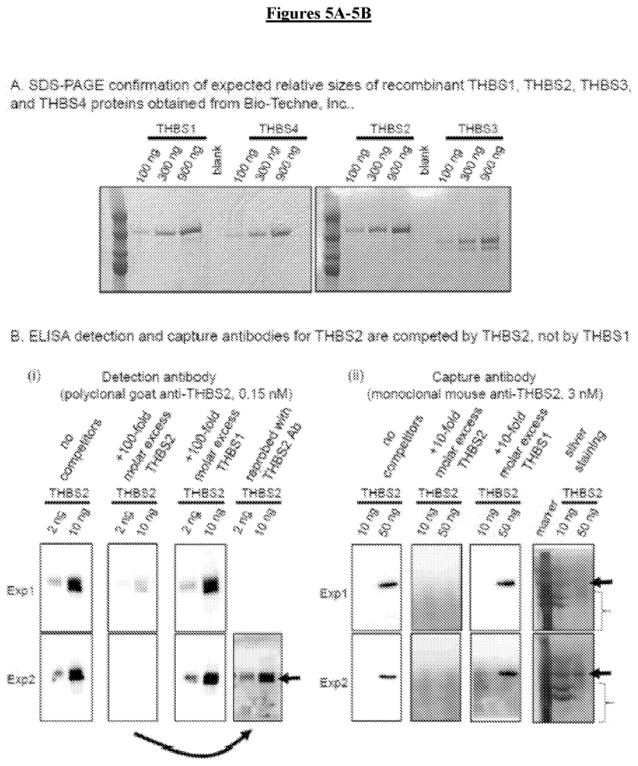

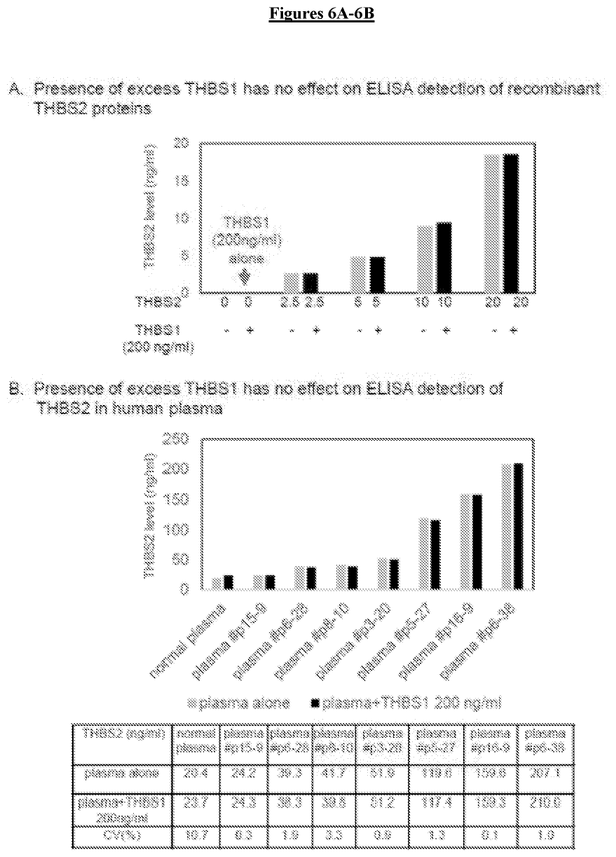

Phase 1 validation: It was examined whether MMP2, MMP10, or THBS2 could discriminate between cancer cases (n=10) and controls (n=10) with an AUC analysis of the sensitivity and the specificity of the markers. All cancer cases for Phase 1 were selected to have CA19-9 concentrations above 55 U/mL which is diagnosed as CA19-9 positive in the clinic. As seen in FIG. 1A, MMP2 was unable to discriminate effectively between cancer cases and controls, and MMP10 signals were undetectable in all plasma samples. By contrast, THBS2 exhibited a c-statistic of 0.76 considering all cases versus controls (n=10) and a c-statistic of 0.886 when considering resectable and locally advanced PDAC (n=7). While human THBS2 has 80% amino acid sequence homology with THBS1, it was demonstrated the specificity of each of the reagents in the THBS2 ELISA assay (FIGS. 5 and 6).

After the Phase 1 validation analysis, a mass spectrometry study of the pooled cancer plasma samples (n=10) and the pooled control plasma samples (n=10) was performed, where the plasma samples were first individually depleted of the 14 most abundant plasma proteins (e.g., serum albumin). At 5% FDR, four unique peptides for THBS2 were identified, of which two were from sequences specific to THBS2 and the other two were from sequences that are conserved between THBS1 and THBS2. One of two peptides specific to THBS2 was present 3-fold greater in the cancer pool compared to the control pool, and another peptide specific to THBS2 was detected only in the cancer pool and not the control pool (Tables 4A & 4B). At 1% FDR, a THBS2-specific peptide was detectable only in the cancer pool (Table 4C). Computational analysis of RNA expression data in TCGA shows that, of all cancers tested, PDAC (n=134) is second to mesothelioma in expressing THBS2 mRNA (FIG. 1B, medians). Taking together the Phase 1 validation by ELISA, mass spectrometry data, and TCGA RNA-seq data, it was concluded that THBS2 merited further study.

Tables 4A-4C: Mass spectrometry of THBS2 concentrations in Phase I plasma samples

TABLE 4A
Peptides searched with pFind 2.8 at 5% FDR

| | | presence of peptides | | area under the curve of | | | | |
|---|---|---|---|---|---|---|---|---|
| | | by search engine | | THBS2 spectrum from | | | | |
| | | pFind 2.8 | | original ms1/ms2 window | | | | |
| | | Pooled | Pooled | Pooled | Pooled | THBS2 | *adjusted | |
| | | normal | PDAC | normal | PDAC | ratio, | ratio of | |
| | | controls | patients | controls | patients | cancer/ | THB32 by | |
| | sequence | (n = 10) | (n = 10) | (n = 10) | (n = 10) | normal | total PSM | FDR |
| peptide 1 | TRNMSACWQDGR | presence | n.d (not- | 55814 | 261090 | 4.66 | 3.04 | FDR 5% |
| (THBS2 | SEQ ID NO: 3 | | determined) | | | | | |
| specific) | | | | | | | | |
| peptide 2 | VCNSPEPQYGGK | n.d | presence | 0 | 792162 | n.d. | n.d. | FDR 5% |
| (THBS2 | SEQ ID NO: 1 | | | | | | | |
| specific) | | | | | | | | |
| peptide 3 | FYWMWK | presence | n.d | 4599455 | 13383548 | 2.9 | 1.88 | FDR 5% |
| (THBS1/ | SEQ ID NO:4 | | | | | | | |
| THBS2 | | | | | | | | |
| shared) | | | | | | | | |
| peptide 4 | NALWHTGNTPGQVR | n.d | presence | 8373610 | 21837763 | 2.6 | 1.69 | FDR 5% |
| (THBS1/ | SEQ ID NO: 2 | | | | | | | |
| THBS2 | | | | | | | | |
| shared) | | | | | | | | |
| Total | | | | 22330 | 34402 | 1.54 | | FDR 5% |
| number | | | | | | | | |
| of PSM | | | | | | | | |
| (peptide | | | | | | | | |
| spectrum | | | | | | | | |
| matches) | | | | | | | | |

*Adjusted ratio of THBS2 was calculated from the original ratio THBS2 dividing by a normalization factor (1.54) acquired by dividing the total number of peptide spectrum matches (PSM) in cancer by the total PSM number in the normal sample. The ratio for peptide 2 and the overall average ratio of THBS2 was not computed because peptide 2 was only observed in the PDAC pooled sample.

TABLE 4B
Peptides searched with pFind3.0 at 5% FDR

| | | presence of peptides by search | | |
|---|---|---|---|---|
| | | engine pFind 3. | | |
| | | Pooled normal | Pooled PDAC | |
| | sequence | controls (n = 10) | patients (n = 10) | FDR |
| peptide 1 (THBS2 | TRNMSACWQDGR | n.d | n.d | FDR 5% |
| specific) | (SEQ ID NO: 3) | | | |
| peptide 2 (THBS2 | VCNSPEPQYGGK | n.d | presence | FDR 5% |
| specific) | (SEQ ID NO: 1) | | | |
| peptide 3 | FYVVMWK | presence | n.d | FDR 5% |
| (THBS1/THBS2 | (SEQ ID NO: 4) | | | |
| shared) | | | | |
| peptide4 | NALWHTGNTPGQVR | presence | presence | FDR 5% |
| (THBS1/THBS2 | (SEQ ID NO: 2) | | | |
| shared) | | | | |

TABLE 4C
Peptides searched with pFind3.0 at 1% FDR

| | | presence of peptides by search | | |
|---|---|---|---|---|
| | | engine pFind 3. | | |
| | | Pooled normal | Pooled PDAC | |
| | sequence | controls (n = 10) | patients (n = 10) | FDR |
| peptide 1 (THBS2 | TRNMSACWQDGR | n.d | n.d | FDR 1% |
| specific) | (SEQ ID NO: 3) | | | |
| peptide 2 (THBS2 | VCNSPEPQYGGK | n.d | presence | FDR 1% |
| specific) | (SEQ ID NO: 1) | | | |
| peptide 3 | FYVVMWK | n.d | n.d | FDR 1% |
| (THBS1/THBS2 | (SEQ ID NO: 4) | | | |
| shared) | | | | |
| peptide4 | NALWHTGNTPGQVR | presence | presence | FDR 1% |
| (THBS1/THBS2 | (SEQ ID NO: 2) | | | |
| shared) | | | | |

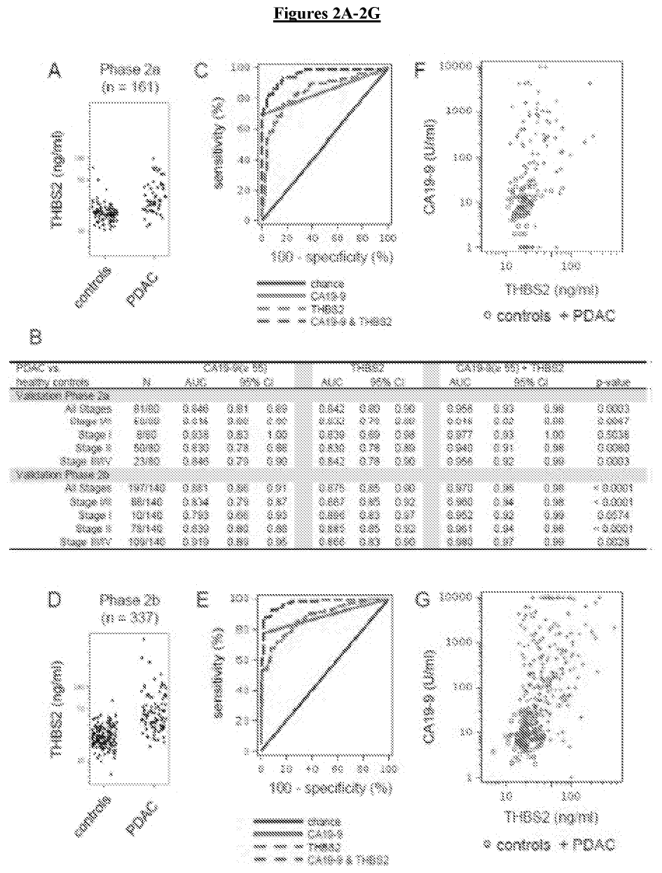

Phase 2a validation: Further validation of THBS2 involved plasma samples in a Phase 2a study (Table 1) that contained CA19-9 negative and positive cases. The median ELISA value for THBS2 at all PDAC stages (N=81) in the Phase 2a group, 29.7 ng/ml, was 12.2 ng/ml higher than observed in controls (N=80) (FIG. 2A), consistent with the mass spectrometry data for Phase 1. THBS2 exhibited a c-statistic of 0.842 for all PDAC samples compared to controls (n=161, FIG. 2B, “All Stages”). In the same sample set, CA19-9 had a comparable c-statistic of 0.846 for all PDAC samples compared to controls (FIG. 2B, “All Stages”).

TABLE 1
Demographic and clinical characteristics of patients whose samples were used in Phases 1, 2a, and 2b.

| | Adenocarcinoma | | Adenocarcinoma | | | | IPMN, no | | | | | |
|---|---|---|---|---|---|---|---|---|---|---|---|---|
| | Stage I/II | | Stage III/IV | | Controls | | Adenocarcinoma | | PNET | | Pancreatitis | |
| Discovery | N = 6 | | N = 4 | | N = 10 | | | | | | | |
| Phase 1 | | | | | | | | | | | | |
| Age | 56.8 | (7.5) | 65.0 | (13.0) | 62.2 | (15.4) | | | | | | |
| Male Gender | 4 | (66.7%) | 2 | (50.0%) | 5 | (50.0%) | | | | | | |
| Body Mass | 31.2 | (8.6) | 26.3 | (5.4) | 26.0 | (3.9) | | | | | | |
| Index (kg/m²) | | | | | | | | | | | | |
| Personal | 1 | (16.7%) | 0 | | 2 | (20.0%) | | | | | | |
| History of | | | | | | | | | | | | |
| Diabetes | | | | | | | | | | | | |
| CA19-9 | 20770.8 | (47060.9) | 111224.0 | (217927.2) | 12.0 | (6.4) | | | | | | |
| Stage of | | | | | | | | | | | | |
| Disease | | | | | | | | | | | | |
| I | 1 | (16.7%) | | | | | | | | | | |
| IIA | 1 | (16.7%) | | | | | | | | | | |
| IIB | 4 | (66.7%) | | | | | | | | | | |
| III | | | 1 | (25.0%) | | | | | | | | |
| IV | | | 3 | (75.0%) | | | | | | | | |
| Validation | N = 58 | | N = 23 | | N = 80 | | | | | | N = 28 | |
| Phase 2a | | | | | | | | | | | | |
| Age | 67.6 | (9.4) | 68.6 | (10.9) | 67.4 | (9.8) | | | | | 61.5 | (9.4) |
| Male Gender | 43 | (74.1%) | 12 | (52.2%) | 54 | (67.5%) | | | | | 19 | (67.9%) |
| Body Mass | 28.9 | (5.2) | 28.5 | (5.7) | 27.2 | (4.7) | | | | | 26.3 | (4.8) |
| Index (kg/m²) | | | | | | | | | | | | |
| Personal | 15 | (25.9%) | 3 | (13.0%) | 12 | (15.0%) | | | | | 3 | (10.7%) |
| History of | | | | | | | | | | | | |
| Diabetes | | | | | | | | | | | | |
| CA19-9 | 305.3 | (411.1) | 2137.5 | (2983.7) | 10.6 | (6.9) | | | | | 68.9 | (165.5) |
| Stage of | | | | | | | | | | | | |
| Disease | | | | | | | | | | | | |
| I | 1 | (1.7%) | | | | | | | | | | |
| IA | 1 | (1.7%) | | | | | | | | | | |
| IB | 6 | (10.3%) | | | | | | | | | | |
| II | 16 | (27.6%) | | | | | | | | | | |
| IIA | 13 | (22.4%) | | | | | | | | | | |
| IIB | 21 | (36.2%) | | | | | | | | | | |
| III | | | 10 | (43.5%) | | | | | | | | |
| | | | 13 | (56.5%) | | | | | | | | |
| Validation | N = 88 | | N = 109 | | N = 140 | | N = 115 | | N = 30 | | N = 55 | |
| Phase 2b | | | | | | | | | | | | |
| Age | 66.5 | (11.3) | 64.4 | (11.0) | 65.8 | (10.8) | 68.8 | (8.7) | 63.2 | (7.1) | 55.9 | (17.7) |
| Male Gender | 45 | (51.1%) | 62 | (56.9%) | 70 | (50.0%) | 58 | (50.4%) | 22 | (73.3%) | 27 | (50.0%) |
| Body Mass | 28.4 | (5.4) | 29.1 | (6.0) | 27.1 | (4.4) | 26.5 | (4.0) | 29.0 | (4.9) | 27.8 | (5.0) |
| Index (kg/m²) | | | | | | | | | | | | |
| Personal | 34 | (38.6%) | 25 | (22.9%) | 15 | (10.7%) | 20 | (17.4%) | 10 | (33.3%) | 7 | (13.0%) |
| History of | | | | | | | | | | | | |
| Diabetes | | | | | | | | | | | | |
| CA19-9 | 633.8 | (1665.9) | 2399.3 | (3481.1) | 12.0 | (14.5) | 15.4 | (12.3) | 45.3 | (98.0) | 35.9 | (66.0) |
| Stage of | | | | | | | | | | | | |
| Disease | | | | | | | | | | | | |
| I | 4 | (4.5%) | | | | | | | | | | |
| IA | 2 | (2.3%) | | | | | | | | | | |
| IB | 4 | (4.5%) | | | | | | | | | | |
| II | 37 | (42.0%) | | | | | | | | | | |
| IIA | 15 | (17.0%) | | | | | | | | | | |
| IIB | 26 | (29.5%) | | | | | | | | | | |
| III | | | 41 | (37.6%) | | | | | | | | |
| IV | | | 68 | (62.4%) | | | | | | | | |

IPMN: Intraductal papillary mucinous neoplasm; PNET: Pancreatic neuroendocrine tumor; Continuous variables (Age, Body Mass Index, and CA19-9 concentration) are presented as mean (standard deviation). Categorical variables (Male Gender, Personal History of Diabetes, and Stage of Disease) are presented as frequency (percentage).

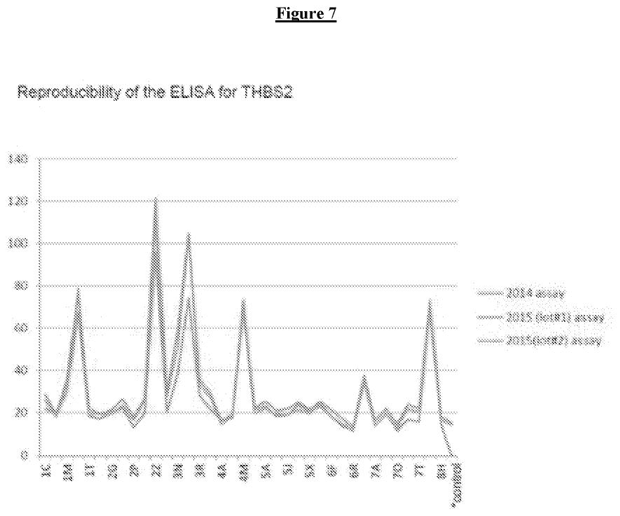

The data for the THBS2 ELISAs were highly reproducible across three different lot numbers tested on the same subset of Phase 2a samples, over a two-year period, with an average 10% coefficient of variation (CV) across the samples (FIG. 7). The samples included 4 plasmas that were re-frozen and thawed twice, and 3 plasma samples that were re-frozen and thawed three times. It was conclude that the THBS2 assay is robust to common differences in plasma sample handling and assessment observed in clinical settings.

To determine if CA19-9 and THBS2 together could constitute a more discriminatory panel than either marker alone, logistic regression to estimate the combined probability of their case discriminatory ability was performed. A combination of CA19-9 and THBS2 for all cases versus controls with the Phase 2a data yielded a c-statistic of 0.956 (95% CI 0.93, 0.98) (FIG. 2B, “All Stages”, 2C), indicating the utility of the two-marker panel.

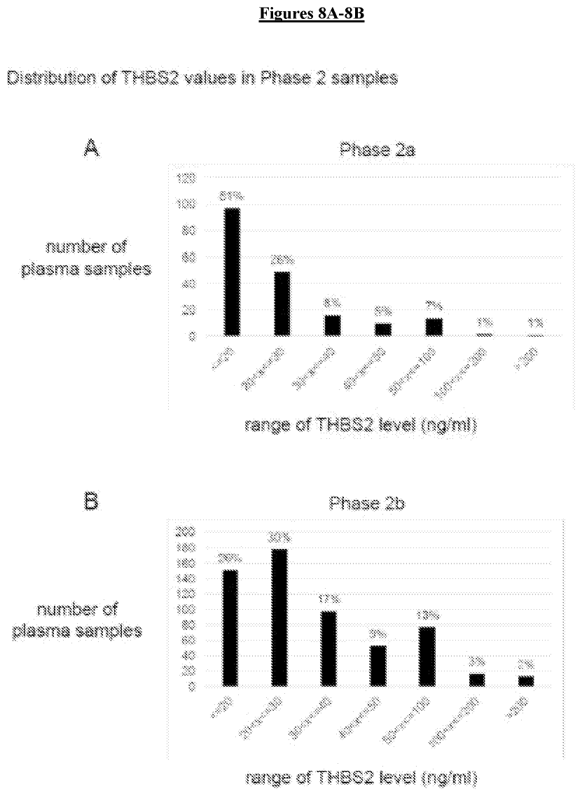

Phase 2b validation studies for a PDAC biomarker panel of CA19-9 and THBS2: An independent Phase 2b validation study (see Table 1 for specimens) was performed with an increased sample size. Temporal validation (18) was accomplished, as the Phase 2b analysis was over one year after that for Phase 2a. The distribution of THBS2 values across the Phase 2a and 2b studies is shown in FIG. 8 and the range and median values of THBS2 and CA19-9 are shown in Table 5.

TABLE 5
Range and median values of THBS2 and CA19-9 in this study

| | n | median | min | P_25 | P_75 | max |
|---|---|---|---|---|---|---|
| THBS2 | | | | | | |
| Adenocar- | 228 | 37.171 | 11.590 | 25.6620 | 60.80 | 451.40 |
| cinoma | | | | | | |
| Stage I/II | 152 | 35.291 | 11.590 | 25.582 | 55.41 | 451.40 |
| 152 | | | | | | |
| Stage III/IV | 136 | 40.797 | 11.699 | 26.621 | 67.10 | 269.71 |
| Controls | 230 | 19.298 | 6.425 | 16.521 | 24.10 | 73.39 |
| IPMN, | 115 | 24.666 | 10.142 | 19.426 | 32.55 | 72.52 |
| no Adeno | | | | | | |
| Islet Cell | 30 | 30.033 | 13.872 | 22.212 | 45.67 | 433.46 |
| Pancreatitis | 83 | 24.094 | 6.458 | 17.949 | 31.74 | 209.12 |
| Ca19-9 | | | | | | |
| Adenocar- | 228 | 219.500 | 0.599 | 55.9000 | 1137.00 | 10000.00 |
| cinoma | | | | | | |
| Stage I/II | 152 | 141.000 | 0.599 | 31.800 | 442.00 | 10000.00 |
| 152 | | | | | | |
| Stage III/IV | 136 | 615.500 | 0.600 | 103.000 | 3973.00 | 10000.00 |
| Controls | 230 | 8.650 | 0.599 | 5.400 | 14.00 | 136.00 |
| IPMN, | 115 | 12.200 | 0.600 | 6.700 | 21.31 | 56.20 |
| no Adeno | | | | | | |
| Islet Cell | 30 | 11.200 | 0.599 | 5.100 | 29.10 | 461.00 |
| Pancreatitis | 83 | 14.700 | 0.600 | 8.500 | 32.20 | 833.00 |

The c-statistics for CA19-9 and THBS2 alone, 0.881 and 0.875, respectively, were slightly better with the larger sample size of Phase 2b (n=337), compared to Phase 2a (n=161), and the combination of the two markers yielded a c-statistic of 0.970 (95% CI=0.96, 0.98) (FIG. 2B, “All Stages”, 2D, E). With regard to the distribution variability, the 75th percentile of the control values falls below the 25th percentile of the cases. Furthermore, the 95th percentile of the controls falls below the median measure observed in the case samples. The fact that 50% of the case values exceed 95% of the control values is likely driving the AUC observed for THBS2 with regard to being able to discern between cases and controls.

Individual and combined marker performance in the Phase 2a and 2b studies at resectable PDAC (stages I, II) and locally advanced and metastatic PDAC (stages III, IV) were compared. Notably, the combination panel of CA19-9 and THBS2 performed well across all stages of PDAC (FIG. 2B).

More detailed analysis of the distribution of ELISA signals provided insight into how the combination of CA19-9 and THBS2 performs so well. As observed in the scatter plots in FIGS. 2F and 2G, various cases (red +) have essentially zero CA19-9 signal (i.e., along the bottom of the plot), consistent with their being likely from PDAC patients who are Lewis antigen negative; however, many of these cases have elevated THBS2 concentrations. Similarly, several cases exhibit THBS2 concentrations that overlap with the upper range of the group of controls, and these cases exhibit high CA19-9 concentrations. Thus, the two markers appear complementary in their ability to detect PDAC.

While stages I, IIA, and IIB are classified as resectable tumors in the 6th Edition AJCC Pancreatic Cancer Staging System (34), only stages I and IIA are considered “early.” Therefore, the AUCs for stage combinations I+IIA+IIB+II (unspecified), I+IIA+II (unspecified), and I+IIA within Phases 2A and 2B of this study were directed compared. The AUC and 95% CI values are comparable for the 2-marker combination across these three subsets, indicating that the exclusion of the “questionable early stage” stage IIB samples has limited impact on marker performance (Table 6).

TABLE 6
Impact of excluding stage IIB (and unspecified stage II) subjects

| | | CA19-9 (≤55) | | | THBS2 | | | CA19-9 (≤55) + THBS2 | | | |
|---|---|---|---|---|---|---|---|---|---|---|---|
| | N | AUC | 95% | CI | AUC | 95% | CI | AUC | 95% | CI | p-value |
| Validation | | | | | | | | | | | |
| Phase 2a | | | | | | | | | | | |
| Stage | 58/80 | 0.845 | 0.80 | 0.89 | 0.832 | 0.78 | 0.89 | 0.946 | 0.92 | .098 | 0.0067 |
| I/IIA/II/IIB | | | | | | | | | | | |
| Stage | 37/80 | 0.851 | 0.80 | 0.91 | 0.810 | 0.74 | 0.89 | 0.937 | 0.90 | 0.98 | 0.0767 |
| I/IIA/II | | | | | | | | | | | |
| Stage I/IIA | 21/80 | 0.905 | 0.85 | 0.97 | 0.819 | 0.73 | 0.91 | 0.919 | 0.85 | 1.00 | 0.7058 |
| Validation | | | | | | | | | | | |
| Phase 2b | | | | | | | | | | | |
| Stage | 88/140 | 0.834 | 0.79 | 0.87 | 0.887 | 0.85 | 0.92 | 0.960 | 0.94 | 0.98 | <0.0001 |
| I/IIA/II/IIB | | | | | | | | | | | |
| Stage | 62/140 | 0.824 | 0.78 | 0.87 | 0.861 | 0.82 | 0.90 | 0.950 | 0.93 | 0.97 | 0.0002 |
| I/IIA/II | | | | | | | | | | | |
| Stage I/IIA | 25/140 | 0.773 | 0.70 | 0.86 | 0.905 | 0.86 | 0.95 | 0.958 | 0.93 | 0.98 | 0.0005 |

In Tables 7A and 7B, the relationship between THBS2 plasma values and age, sex, and presence of diabetes mellitus in the cohort was evaluated. It was observed no apparent associations of these parameters for any of the diagnosis groups of PDAC Adeno UII, Adeno III/IV, pancreatitis, IPMN, insulinoma (“islet cell”), and healthy controls. As described further below, the relationship to jaundice, a common clinically observed sign of pancreatic cancer was also observed, and similarly found few differences. Given the overall lack of association, any of these factors were not included as adjustor variables in subsequent modeling.

TABLE 7A
THBS2 values by sex, and Diabetes Mellitus (DM) status

| group | | n | median | min | P_25 | P_75 | max |
|---|---|---|---|---|---|---|---|
| IPMN | Female | 57 | 25.8860 | 12.096 | 20.7010 | 32.2340 | 70.881 |
| IPMN | Male | 58 | 24.1590 | 10.142 | 18.5610 | 33.2390 | 72.515 |
| PNET | Female | 8 | 29.3040 | 21.391 | 24.1965 | 45.8290 | 100.724 |
| PNET | Male | 22 | 32.7955 | 13.872 | 19.1610 | 45.6740 | 433.460 |
| Pancreatitis | Female | 36 | 21.0220 | 6.458 | 17.6355 | 30.4205 | 67.388 |
| Pancreatitis | Male | 46 | 25.2530 | 11.409 | 19.0730 | 36.3010 | 209.117 |
| Adenocarcinoma | Female | 60 | 38.0070 | 15.182 | 30.2070 | 61.8390 | 216.687 |
| Stage I/II | | | | | | | |
| Adenocarcinoma | Male | 92 | 31.8888 | 11.590 | 22.5440 | 51.6485 | 451.397 |
| Stage I/II | | | | | | | |
| Adenocarcinoma | Female | 60 | 40.5035 | 15.698 | 26.2460 | 67.0990 | 218.449 |
| Stage III/IV | | | | | | | |
| Adenocarcinoma | Male | 76 | 40.8955 | 11.699 | 27.1895 | 67.2140 | 269.711 |
| Stags III/IV | | | | | | | |
| Controls | Female | 101 | 20.7900 | 12.012 | 17.2330 | 26.2680 | 65.573 |
| Controls | Male | 129 | 18.1210 | 6.425 | 15.4790 | 22.3270 | 73.392 |
| IPMN | No DM | 95 | 23.8800 | 10.142 | 19.3670 | 29.9620 | 60.287 |
| IPMN | DM | 20 | 34.1695 | 13.764 | 24.6135 | 51.5630 | 72.515 |
| PNET | No DM | 20 | 28.6720 | 13.872 | 20.2760 | 62.8445 | 433.460 |
| PNET | DM | 10 | 39.3765 | 13.967 | 23.1640 | 44.9590 | 111.390 |
| Pancreatitis | No DM | 72 | 24.5510 | 6.458 | 18.1123 | 30.4205 | 209.117 |
| Pancreatitis | DM | 10 | 26.1715 | 11.637 | 18.4470 | 42.5880 | 77.424 |
| Adenocarcinoma | No DM | 102 | 35.0490 | 11.590 | 25.5830 | 57.6440 | 248.952 |
| Stage I/II | | | | | | | |
| Adenocarcinoma | DM | 50 | 37.2575 | 15.599 | 23.2920 | 53.7950 | 451.397 |
| Stage I/II | | | | | | | |
| Adenocarcinoma | No DM | 108 | 38.7715 | 11.699 | 27.7875 | 68.5970 | 269.711 |
| Stage III/IV | | | | | | | |
| Adenocarcinoma | DM | 28 | 43.7983 | 15.698 | 23.8990 | 65.1340 | 238.642 |
| Stage III/IV | | | | | | | |
| Controls | No DM | 201 | 19.2820 | 6.425 | 16.5060 | 23.6850 | 65.573 |
| Controls | DM | 29 | 19.3320 | 9.654 | 17.0210 | 24.4930 | 73.392 |

TABLE 7B
Spearman Correlation analysis of age and THBS2 values

| group | Spearman Correlation |
|---|---|
| IPMN | −0.00401 |
| PNET | −0.15872 |
| Pancreatitis | −0.02588 |
| Adenocarcinoma Stage I/II | −0.08581 |
| Adenocarcinoma Stage III/IV | −0.11019 |
| Controls | 0.06188 |

Establishing a provisional cutoff point for THBS2 for clinical use: To determine a THBS2 plasma concentration to use as a cutoff point for discriminating healthy versus PDAC cases in the clinic, the distribution of THBS2 values based upon the 230 healthy controls from the combined Phase 1, 2a, and 2b studies was first considered. From this distribution, six cutoffs that represented a range of approximate false positive rates from 0 to 5 percent were chosen. These cutoffs were then evaluated for their sensitivity in detecting PDAC in the Phase 2a and 2b samples. As seen in Table 2 for the Phase 2b study, a concentration of THBS2 at or above 42 ng/ml detects about half of the PDAC cases (sensitivity) with 99% specificity. Combining the conventional CA19-9 cutoff of >55U/m1 and a cutoff of 42 ng/ml THBS2 in the Phase 2b samples, it was observed 98% specificity and 87% sensitivity.

TABLE 2
THBS2 cut points based upon percentiles of distribution in controls

| | | | Phase 2a | | Phase 2b | |
|---|---|---|---|---|---|---|
| Marker | | Cutoff | Sensitivity | Specificity | Sensitivity | Specificity |
| CA19-9(≥55) | | | 69.14 | 100 | 77.66 | 98.57 |
| THBS2 (ng/ml) | | | | | | |
| | 95% | 36 | 33.33 | 96.25 | 58.38 | 93.57 |
| | 96% | 37 | 33.33 | 97.50 | 57.36 | 95.00 |
| | 97% | 38 | 30.86 | 97.50 | 53.81 | 96.43 |
| | 98% | 40 | 28.40 | 97.50 | 53.30 | 97.86 |
| | 99% | 42 | 24.69 | 97.50 | 51.78 | 99.29 |
| | 100% | 73.4 | 7.41 | 100 | 23.35 | 100 |
| CA19-9(≥55) & | | | | | | |
| THBS2 (ng/ml) | | | | | | |
| | 95% | 36 | 74.07 | 96.25 | 88.32 | 92.86 |
| | 96% | 37 | 74.07 | 97.50 | 88.32 | 94.29 |
| | 97% | 38 | 74.07 | 97.50 | 87.82 | 95.71 |
| | 98% | 40 | 74.07 | 97.50 | 87.82 | 97.14 |
| | 99% | 42 | 72.84 | 97.50 | 87.31 | 97.86 |
| | 100% | 73.4 | 69.14 | 100 | 81.73 | 98.57 |

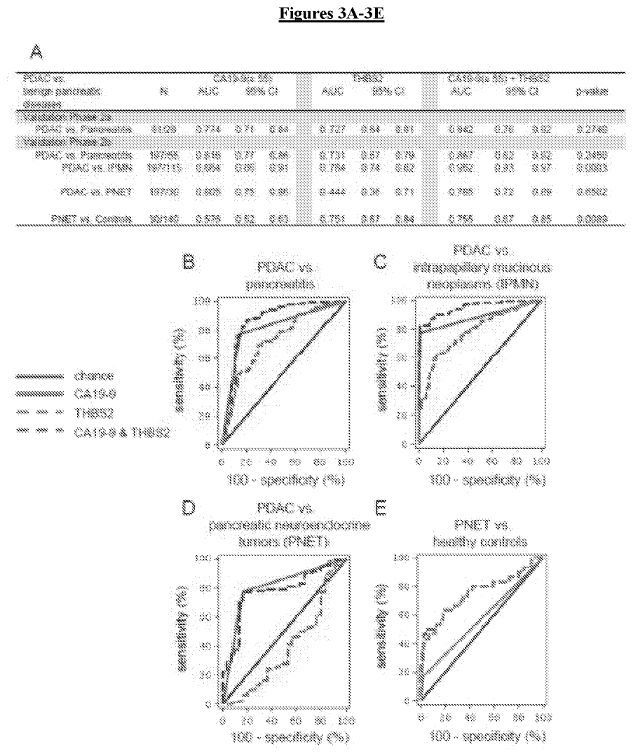

Comparisons of THBS2/CA19-9 panel against other benign pancreatic conditions: As noted in FIG. 3A, B, when considering all PDAC cases versus chronic pancreatitis (Phase 2a, n=109; Phase 2b, n=252), c-statistics including CA19-9 increased from 0.774 or 0.816 (alone) to 0.842 or 0.867 (with THBS2) considering the Phase 2a or Phase 2b data, respectively. The THBS2/CA19-9 panel performed well to discriminate all PDAC cases tested (stages I-IV) versus intraductal papillary mucinous neoplasms (IPMN) (N=312), with a c-statistic of 0.952 (FIG. 3A, C). Thus, the THBS2/CA19-9 panel can distinguish PDAC from IPMN, and it helps to distinguish PDAC from pancreatitis, compared to CA19-9 alone.

THBS2 lacked the ability to discriminate between all PDAC cases and pancreatic neuroendocrine tumors (PNET) and hindered, rather than enhanced, the c-statistic of CA19-9 (FIG. 3A, D). Considering a lack of markers available for PNET and poor performance of THBS2 to discriminate PDAC from PNET, it was examined whether THBS2 can discriminate PNET (N=30) from healthy normal controls (N=149). CA19-9 alone did not discriminate PNET samples, as previously reported (35). However, THBS2 could discriminate PNET from healthy normal controls, with a c-statistic 0.751 (FIG. 3A, E).

PDAC can result in obstructive jaundice that can confound plasma assays (20, 36). Of the 288 adenocarcinoma cases included in these studies, clinical total serum bilirubin information for 279 cases (96.9%) (Table 8A) was retrieved. Of the 279 with such information, 70 (25.1%) were inferred to have obstructive jaundice, based on total bilirubin concentrations being ≥3.5 mg/dl. Slightly lower median CA19-9 concentrations (208.5 vs 220) as well as elevated median THBS2 concentrations (56.4 vs 33.0) were observed in PDAC subjects with jaundice, when compared to those without jaundice, indicating that obstructive jaundice influenced both CA19-9 and THBS2 concentrations. Yet 14 out 55 (25%) of PDAC patients with normal CA19- 9 and without jaundice have elevated THBS2 (≥42) (Table 8B, line 3). Also, 8 out of 13 (62%) of patients with normal CA19-9 and with jaundice showed elevated THBS2 (≥42) (Table 8B, line 5). Therefore, THBS2 identifies a subset of non-jaundice adenocarcinoma cases with normal CA19-9 concentrations. Furthermore, stratifying the biomarker panel performance by overall PDAC or PDAC without jaundice, versus controls, in the Phase 2a and 2b studies affected the AUCs by less than 0.01, which was consider negligible (Table 8C). Due to limited availability of benign biliary disease samples, THBS2 and CA19-9 concentrations between benign biliary disease, non-jaundice PDAC, and jaundice PDAC have not been compared.

TABLE 8A
Obstructive jaundice cases in the PDAC cohorts

Obstructive Jaundice (Total Bilirubin ≥ 3.5)

| | | | |
|---|---|---|---|
| Unknown | No | Yes | Total |

| Stage | N | N | % | N | % | N |
|---|---|---|---|---|---|---|
| I | 0 | 4 | 80.0 | 1 | 20.0 | 5 |
| IA | 0 | 0 | 0.0 | 3 | 100.0 | 3 |
| IB | 0 | 8 | 80.0 | 2 | 20.0 | 10 |
| II | 2 | 42 | 80.77 | 10 | 19.23 | 52 |
| IIA | 1 | 16 | 57.14 | 12 | 42.86 | 28 |
| IIB | 2 | 36 | 73.47 | 13 | 26.53 | 49 |
| III | 3 | 35 | 71.43 | 14 | 28.57 | 49 |
| IV | 1 | 68 | 81.93 | 15 | 18.07 | 83 |
| Total | 9 | 209 | | 70 | | 279 |

TABLE 8B
THBS2 and CA19-9 values and obstructive jaundice status

| | | Ca19-9 | | THBS2 Clean | | Ca19-9, THBS2 | |
|---|---|---|---|---|---|---|---|
| | N | AUC | 95% CI | AUC | 95% CI | AUC | 95% CI |
| Phase 2a PDAC | 81/80 | 0.846 | 0.81, 0.88 | 0.843 | 0.80, 0.89 | 0.955 | 0.93, 0.98 |
| Phase 2a PDAC- | 13/80 | 0.923 | 0.83, 1.00 | 0.931 | 0.87, 0.99 | 0.977 | 0.94, 1.00 |
| Jaundice | | | | | | | |
| Phase 2a PADAC - | 65/80 | 0.831 | 0.78, 0.88 | 0.819 | 0.76, 0.88 | 0.950 | 0.92, 0.98 |
| No Jaundice | | | | | | | |
| Phase 2b PDAC | 197/140 | 0.881 | 0.86, 0.90 | 0.875 | 0.85, 0.90 | 0.970 | 0.96, 0.98 |
| Phase 2b PDAC- | 54/140 | 0.891 | 0.85, 0.94 | 0.974 | 0.96, 0.99 | 0.991 | 0.98, 1.00 |
| Jaundice | | | | | | | |
| Phase 2b PDAC- | 138/140 | 0.873 | 0.84, 0.90 | 0.837 | 0.80, 0.87 | 0.962 | 0.962, 0.98 |
| No Jaundice | | | | | | | |

TABLE 8C
AUC values for CA19-9, THBS2, and combined markers by jaundice
status in Phases 2a and 2b of PDAC cases versus Controls.

| | | THBS2 | | | | | |
|---|---|---|---|---|---|---|---|
| Obstructive | | <42 | | ≥42 | | Total | |
| Jaundice | Ca19-9 | N | % | N | % | N | % |
| Unknown | <55 | 1 | 100 | 0 | 0 | 1 | 11 |
| Unknown | ≥55 | 6 | 75 | 2 | 25 | 8 | 89 |
| No | <55 | 41 | 75 | 14 | 25 | 55 | 25 |
| No | ≥55 | 96 | 62 | 58 | 38 | 154 | 74 |
| Yes | <55 | 5 | 38 | 8 | 62 | 13 | 19 |
| Yes | ≥55 | 13 | 23 | 44 | 77 | 57 | 81 |

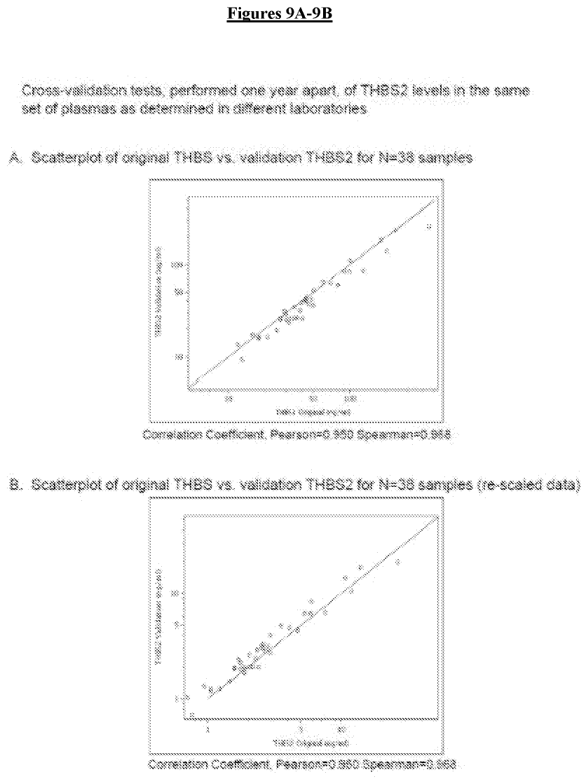

Cross-validation studies: Following these analyses, an independent biomarker development laboratory at the University of Pennsylvania tested a subset of the Phase 2b samples for THBS2 concentrations. Thirty-eight samples were randomly selected to cover the entire range of THBS2 concentrations, focusing on those around the cutoff value. The samples were de-identified and provided without communication other than the manufacturer's instructions for the ELISA assay and the methods section of this paper. The ELISA assays for THBS2 were performed over a year later than the original study and with different lot number reagents. As seen in FIG. 9A, the THBS2 concentrations in original and cross-validated assays were highly concordant and yielded Pearson and Spearman correlation coefficients of 0.95 and 0.968, respectively.

It was noticed that the THBS2 signals were slightly lower in the cross-validated data, including for the human normal control plasma used on each plate. The original studies, all performed in the Zaret laboratory, yielded an average value of 17 ng/ml for the normal control plasma, whereas the cross-validation study yielded a value of 13.25 for the normal control plasma. The lower overall values of unknowns caused 4 of the 38 samples that were just over the 42 ng/ml cutoff, to fall below the cutoff (Table 9A).

To accommodate for operational differences, a scalar was created where the original 42 ng/ml cutoff was divided by the original 17 ng/ml average, normal plasma control value, to yield a scalar cutoff of 2.47. The THBS2 result was therefore divided for each unknown in the cross-validation study by the value (13.25) of the normal control plasma. Scaling does not affect the correlation coefficient (FIG. 9B). With the data scaled in this fashion, two samples that were below the 42 ng/ml cutoff in the original samples were now above the cutoff in the cross-validation data (Table 9B). Thus while the scaling method improves the outcome of the cross-validation assay, careful calibration is needed to ensure consistency in the assay results over time and with different batches of reagent, once a cutoff for clinical practice is determined.

TABLE 9A
Cross tabulation of normal vs. elevated THBS2 values,
given a 42 ng/ml cutoff, for the original and cross-
validation THBS2 assays (Kappa = 0.786)

| | THBS2 Validation | | |
|---|---|---|---|
| THBS2 | <42 | ≥42 | Total |
| <42 | 20 | 0 | 20 |
| ≥42 | 4 | 14 | 18 |
| Total | 24 | 14 | 38 |

TABLE 9B
Cross tabulation of normal vs. elevated scaled THBS2
values, given a cutoff of 2.47, for the original
and cross-validation THBS2 assays (Kappa = 0.895)

| | THBS2 Validation | | |
|---|---|---|---|
| THBS2 | <2.47 | ≥2.47 | Total |
| <2.47 | 18 | 2 | 20 |
| ≥2.47 | 0 | 18 | 18 |
| Total | 18 | 20 | 38 |

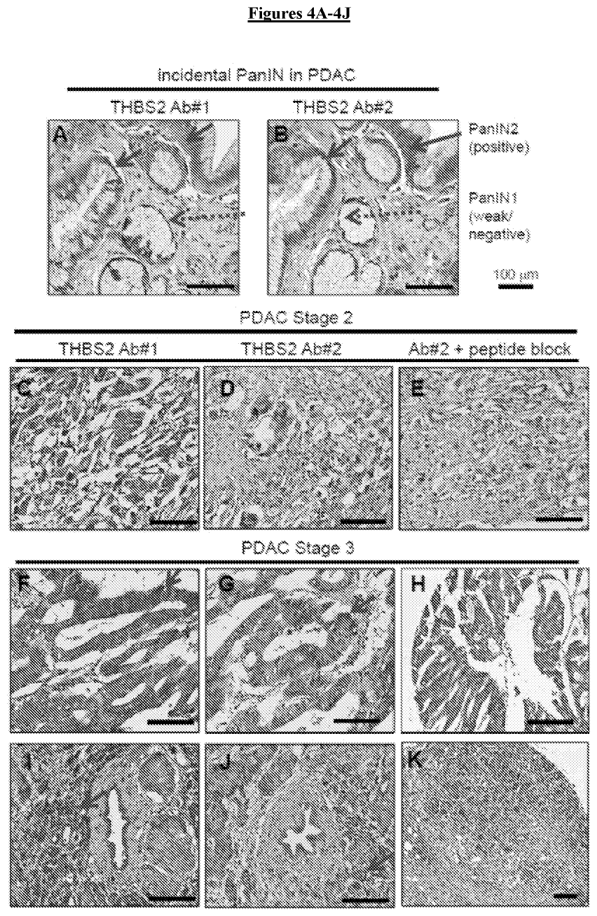

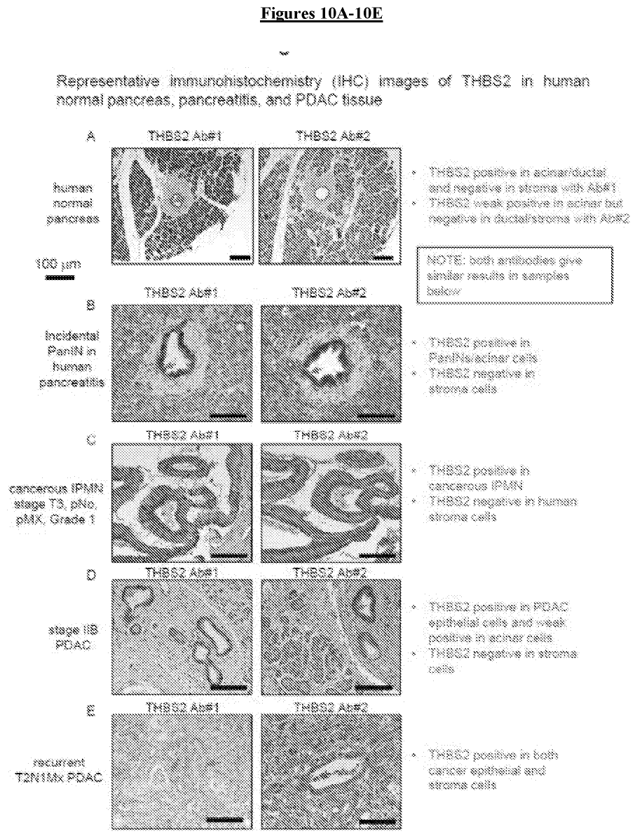

Expression of THBS2 in human PDAC: Immunohistochemistry was preformed to determine the cells expressing THBS2 in a total of 42 cases of human PDAC and 4 cases of incidental PanIN and IPMN by. All 42 cases of PDAC and all 4 cases of incidental PanIN/IPMN exhibited detectable THBS2 (FIG. 4; FIG. 10, Table 10). Two different antibodies detected THBS2 in PanIN2 epithelia occurring incidentally in PDAC, but barely in PanIN1 epithelia (FIG. 4A, B). Both antibodies also detected THBS2 in Stage II and Stage III PDAC, and a 10-fold excess of peptide specific to the second antibody blocked the signals to that antibody (FIG. 4C-K). Epithelial cells, but not stromal cells, were predominantly labeled with THBS2 in PanIN/IPMN tissue (4 out of 4) (FIGS. 4A-4B, FIGS. 10B-10C). In PDAC, 32 cases were labeled with THBS2 in epithelial cells, 21 cases were labeled in both epithelial and stromal cells, and in 8 cases the staining was mostly in stromal cells of poorly differentiated PDAC (Table 10). These conclusions are limited by the portion of tissue available from each of the resections, and it is presently unknown how cellular expression relates to secretion or release into the blood. Taken together, the data indicate that the plasma THBS2 concentration is a marker for early stage PDAC.

TABLE 10
Summary of THBS2 immunohistochemistry in a total of 42 human PDAC
and 4 cases of incidental PanIN and IPMN by immunohistochemistry

| | | | % THBS2 | % THBS2 |
|---|---|---|---|---|
| | | | positive in | positive in |
| | PDAC | PanINs/IPMN | PDAC | PanINs/IPMN |
| total (N) | 42 (tissue array | 4 (FCCC) | 100 | 100 |
| | n = 38, FCCC n = 4) | | | |
| THBS2 positive | 42 (tissue array | 4 (FCCC) | 100 | 100 |
| sections | n = 38, FCCC n = 4) | | | |
| the number of | 32 (tissue array | 4 (FCCC) | 76 | 100 |
| sections THBS2 | n = 30, FCCC n = 2) | | | |
| positive in epithelial | | | | |
| cells of PDAC or | | | | |
| PanINs/IPMN | | | | |
| the number of | 21 (tissue array | 0 (FCCC) | 50 | 0 |
| sections THBS2 | n = 19, FCCC n = 2) | | | |
| positive in both | | | | |
| epithelial and | | | | |
| fibroblast cells | | | | |
| THBS2 positive in | 8 (tissue array | 0 (FCCC) | 19 | 0 |
| only poorly | n = 8, FCCC n = 0) | | | |
| differentiated PDAC | | | | |
| or fibroblast cells | | | | |

FCCC = samples from the repository at the Fox Chase Cancer Center

Discussion

With a 5-year survival of stage I PDAC at least four times that of overall PDAC survival rates (34, 37), the THBS2/CA19-9 marker panel may help to detect resectable tumors and improve the prognosis of PDAC. The performance of THBS2 in early stage cancer may relate to the discovery of the marker as being secreted or released from live human PanIN organoids(25), reflecting the value of the iPS reprogramming-based system. While most PDAC patients are diagnosed at advanced stages, it has been proposed that the time from the occurrence of the initiating mutation to the birth of PDAC founder cells (38) can be a decade, suggesting that there is a time to identify developing disease before PDAC can be clinically imaged. PanlNs have been identified in pancreas up to 10 years before the development of infiltrating PDAC (39), underscoring the importance of early diagnosis; though it was recognize that PanINs are observed in the absence of PDAC as well.

It was felt that high specificity outweighs considerations of increased sensitivity because of heightened anxiety in patients over suspected pancreatic cancer plus the costs of subsequent diagnostic evaluation. It was found that with a THBS2 cutoff concentration of 42 ng/ml, THBS2 can discriminate PDAC patients from healthy primary care control with a specificity of 99% (1% FPR) and a sensitivity of 52%. Impressively, combining CA19-9 (>55 U/ml) with THBS2 (>42ng/ml) shows a specificity of 98% and sensitivity of 87% in the larger Phase 2b study. An important strength of this study was the ability to construct large, defined biospecimen sets obtained from a single institution, following standardized processing protocols, a challenge in other validation studies (23).

Decreased Thrombospondin-1 concentrations by mass spectroscopy analysis have been reported in plasmas of PDAC patients and pre-diagnostic samples (20, 40). Jenkinson et al. found that reduced concentrations of THBS-1 occur significantly in PDAC patients with diabetes, but not in PDAC patients without diabetes (20). In contrast, it was observed that elevated concentrations of THBS2 associate with PDAC and found no association of elevated THBS2 concentrations with diabetes mellitus, age, or sex (Tables 7A- 7B). THBS-1 and THBS-2 share 80% of their protein sequence, but have diverged in function and in their genetic regulation (41, 42). It has been shown that elevated THBS2 does not correspond to THBS1 in PDAC. First, the antibodies enclosed in ELISA kit used for the study have negligible cross-reactivity or interference with THBS1 (FIGS. 5A-5B, and FIGS. 6A-6B). Second, it was confirmed that the peptides specific to THBS2 are more abundant in cancer patient plasmas than in normal plasmas, by mass spectrometry (Tables 4A-4C). Thus, elevated THBS2 concentrations in PDAC are independent of the THBS1 concentrations reported in the literature.

A group of scientists has initiated the STARD (Standards for Reporting of Diagnostic Accuracy) with guidelines to improve the reporting of diagnostic accuracy (45). It will be useful to follow these standard guidelines in the clinic by reporting imprecision as the coefficient variation (CV%) and precision as 95% confidence interval near clinical decision points as obtained by repeating the test over several independent days. Also, to reduce even small differences in the assay occurring between different laboratories, presenting the likelihood ratio with 95% CI along with specificity and sensitivity at several cut-off points would be recommended. This cross-validation study was an initial attempt to address these issues and more work is needed for a determination of clinical decision points with confidence.

The combination of THBS2 and CA19-9 improved the discrimination of PDAC from chronic pancreatitis. THBS2 can be effective for diagnosing PNET, where CA19-9 is not applicable, and other cancers with high THBS2 mRNA expression (FIG. 1B). This current report also suggests the utility of THBS2/CA19-9 in both jaundice and non-jaundice pancreatic cancer.

The prevalence of PDAC in different populations affects the positive and negative predictive values for determining the utility of a biomarker in a population. The positive predictive value (PPV) is the probability that subjects with a positive screening test have the disease, and the negative predictive value (NPV) is the probability that subjects with a negative screening test do not have the disease. Given the low prevalence of 4-12.4 cases of pancreatic cancer per 100,000 in the general population (https://seer.cancer.gov/statfacts/html/pancreas.html), the current marker panel with a combined 98% specificity and 87% sensitivity would have a PPV of 0.002, yet with a NPV of 1.0 (2). Yet when viewed in terms of the 1.5% lifetime risk of PDAC in the general population (2), the positive predictive value becomes about 0.4 with an NPV of 0.99. For patients older than 55 years who are newly diagnosed with diabetes (46), with a prevalence of 1% in the general population for PDAC, the PPV is 0.31 and the NPV is 1.0. For first degree relatives of PDAC patients and smokers in the general population, each group with a lifetime risk of 3.75% (47), the PPV is 0.63 and NPV is 0.99. For carriers with relevant germline mutations (in aggregate, including BRCA1, BRCA2, CDKN2A, PALB2), lifetime risk is 40% (47) and the PPV rises to 0.97 and NPV is at 0.92. Therefore, THBS2/CA19-9 marker panel could serve as a low cost, non-intervention screening tool in asymptomatic individuals who have a high risk of developing PDAC (3, 47, 48), or in patients who are newly diagnosed with type 3c diabetes mellitus (49). Therefore, this biomarker panel should be useful for high-risk populations.

Materials and Methods

Study Design and Populations

All procedures were performed using a recommended biomarker phased design following the PRoBE criteria (28, 29). De-identified human plasma samples from the Mayo Clinic pancreas research biospecimen repository were shipped to the lab, which performed ELISA analyses blinded to disease status, and then returned coded data to the Mayo Clinic team for statistical analysis and interpretation.

Collection of biospecimens was approved by the Mayo Clinic Institutional Review Board. Following rapid case finding (50) and informed consent, participants with PDAC provided venous blood samples prior to initiation of cancer therapy. Samples were frozen at −80° C. until used. Similarly, blood samples were obtained from Mayo Clinic through primary care (healthy controls) and gastroenterology clinics (participants diagnosed with chronic pancreatitis, intraductal papillary mucinous neoplasm (IPMN), and pancreatic neuroendocrine tumor (PNET). An aliquot of serum was assayed for CA19-9 (Roche) at the Mayo Clinic Immunochemical Core Laboratory using clinical protocols. Demographic and clinical characteristics in each group are shown in Table 1.

Exploratory Set (Phase 1): Plasma samples from 20 non-Hispanic Caucasian subjects recruited at Mayo Clinic included 10 healthy primary care controls and 10 (6 early stage (I/II), 4 late stage (III/IV)) clinically and/or histologically proven PDAC patients. All cancer cases for Phase 1 were selected to have CA19-9 concentrations above 55 U/mL.

Validation Set (Phase 2a): Plasma samples from 189 non-Hispanic Caucasian subjects recruited at Mayo Clinic included 81 (58 early stage, 23 late stage) clinically and/or histologically proven PDAC patients, 80 healthy primary care controls, and 28 patients with personal history of chronic pancreatitis; patients with hereditary pancreatitis were excluded given their increased risk for PDAC. The controls were matched to the cases by age and sex. Approximately 15% of the healthy controls self-reported a personal history of diabetes.

Validation Set (Phase 2b): Plasma samples collected from 537 non-Hispanic Caucasian subjects recruited at Mayo Clinic included 197 (88 early stage, 109 late stage) clinically and/or histologically proven PDAC patients, 140 healthy primary care controls, 115 patients with IPMN without PDAC, 30 patients with PNET, and 55 patients with a self-reported personal history of chronic pancreatitis; patients with hereditary pancreatitis were excluded. Approximately 11% of the controls self-reported a personal history of diabetes.

Measurement of Biomarkers in Human Plasma

After the ELISA assays for the Phase 1 study was completed, the remaining PDAC (n=10) and control samples (n=10) were each separately depleted of abundant serum proteins by filtration and then High Performance Liquid Chromatography (HPLC) using a Seppro IgY14 LC 10 column (Sigma Aldrich). The resulting 10 samples of cancer plasmas, depleted of abundant proteins, were pooled separately from a pool of the controls and the two pools were subjected to 2D Strong Cation Exchange Chromatography (SCX)/tandem mass spectrometry analysis as previously described (51). In brief, aSCX tip column was made with a 200 ul tip packed with 20 ul PolySULFOETHYL resin (Nest Group). The SCX tip was pre-washed with buffer B (500 mM KC1, 10mM NaH2PO4, 30% acetonitrile, pH 2.6), followed by equilibration with buffer A (10 mM NaH2PO4, 30% acetonitrile, pH 2.6). The lyophilized digested peptides (100 ug) were reconstituted in 50 pi buffer A. The reconstituted digested peptide solution was loaded into the SCX tip column twice, followed by washing with 50 μl buffer A. All flow-through fractions were combined (“flow-through”). The following 100 μl KCl concentration buffers, made by mixing the different proportions of buffer A and B, were used to successively wash the column: 30 mM, 40 mM, 50 mM, 60 mM, 70 mM, 85 mM, 100 mM, 150 mM and 500 mM. A total of 10 fractions was dried and de-salted using the homemade C18 Stage Tips. About 3 μg digested peptides were injected into a 75 μm I.D. X 25cm C18 column with a pulled tip. Easy nLC 1000 was run at 300 nl/min flow rate for 180 min gradient. Online nanospray was used to spray the separated peptides into an Orbitrap Fusion Tribrid mass spectrometer (Thermo Electron). The raw data were acquired with Xcalibur and pFind2.8 software was used to search human Uniprot database. A 5% false discovery rate for the protein spectrum measurement (PSM) was used initially to filter the peptide search results. Table 4 shows the results for a total of four THBS2 peptides that were detected in the pooled cancer plasma samples. The pFind2.8 search engine revealed two unique peptides in each of the pooled plasmas; the cancer pool had a peptide specific THBS2 (VCNSPEPQYGGK, SEQ ID NO: 1) and a peptide shared between THBS1 and THBS2 (NALWHTGNTPGQVR, SEQ ID NO: 2) and the normal pool had a peptide specific to THBS2 (TRNMSACWQDGR, SEQ ID NO: 3) and a peptide shared between THBS1 and THBS2 (FYVVMWK, SEQ ID NO: 4) (Table 4A). THBS2 levels in each of the pooled plasmas were quantified by measuring the area under the peptide signals based on mass and retention time in original ms 1 and ms2 windows. It was then normalized to the total spectral counts in each sample (Table 4A).

To more stringently assess THBS2 peptide levels, it was searched with 1% and 5% FDR settings against the Uniprot Human database (89,796 entries in total) with the updated pFind3.0 search engine (52). Search parameters were set for a precursor mass tolerance of ±7 ppm, fragment mass tolerance of ±0.4 Da, trypsin cleaving after lysine and arginine with up to 2 miscleavages, carbamidomethyl [C]/+57.021 as the fixed modification, and acetyl [proteinN-term]/+42.011, deamidated [NQ]/+0.984, and oxidation [M]/+15.995 as the variable modifications. The target-decoy approach was used to filter the search results, in which the false discovery rate (FDR) was less than 1% or 5% at both the peptide and protein level. At both 1% and 5% FDR, the peptide specific to THBS2 (VCNSPEPQYGGK) and the peptide shared between THBS1 and THBS2 (NALWHTGNTPGQVR) was seen in the cancer pooled sample, consistent with the original pFind2.8 search (Tables 4B and 4C). However, at either 1% or 5% FDR with the pFind3.0 search, no peptides specific to THBS2 sequence were identified and only peptides shared between THBS1 and THBS2 were identified in the normal pooled sample (Tables 4B and 4C). Therefore, more stringent analysis verified THBS2 sequence in cancer pooled sample.

ELISA kits for human MMP2 (Millipore), human MNIP10 (Ray Biotech), and human Thrombospondin-2 Quantikine (DTSP20, R&D systems) were used as described by the manufacturers' instructions. Duplicate 5 μl plasma samples were diluted 10 fold with calibrator diluent RDSP buffer and all 50 μl used for THBS2. Marker concentrations were determined from standard curves of positive control proteins from the kits with a 4 parameter logistic nonlinear regression model using SoftMax Pro Software (Molecular Device). Normal pooled human plasma (IPLA-N, Innovative Research) was tested in duplicate on each ELISA plate. Across 15 independent ELISA plate assays, THBS2 in duplicate control samples of commercial normal pooled human plasma ranged between 15 and 21 ng/ml, with a coefficient of variation of 13%. Also, the inclusion (or exclusion) of occasional plasma samples that were orange or reddish in color, indicating hemolysis, had a negligible impact on the data.

RNA-Seq analysis from the Cancer Genome Atlas (TOGA)

THBS2 mRNA amounts were assessed in TGCA RNA-Seq datasets (http://cancergenome.nih.govi) using the cBioPortal for Cancer Genome (53, 54). Data were downloaded from the UCSC Xena data hub and sample IDs curated using the Broad Institute's Genome Data Analysis Center Firehose. THBS2 mRNA values were estimated by the RSEM algorithm (55), and log 2 (RSEM+1) transformed for FIG. 1B, as parsed and plotted using scripts in Python, R.

Western Blot and ELISA for Validation of THBS2 ELISA Kits for Cross-Reactivity with THBS1

The recombinant THBS proteins were obtained from R&D systems and performed western blot with polyclonal goat anti-THBS2 (detection antibody, working conc 0.15 nM) and monoclonal mouse anti-THBS2 (capture antibody, working conc. 3 nM) to check the cross-reactivity. A detection antibody and a 100-fold molar excess of recombinant THBS1 or THBS2 proteins were incubated in 5% non-fat milk for 30 min at RT for competition assay. The incubated solution was centrifuged at 10K RPM for 15 min to remove any immunocomplexes prior to applying onto a PVDF membrane a total of 2, 10 ng proteins were transferred for detection antibodies. For competition assay of capture antibody, a 10-fold molar excess of recombinant THBS1 or THBS2 proteins with incubated with capture antibody in 5% BSA for 30 min at RT. The incubated solution was centrifuged at 15K RPM for 15 min to remove any immunocomplexes prior to applying onto a PVDF membrane a total of 10, 50 ng proteins were transferred for detection antibodies. The presence of THBS2 in a gel were confirmed by silver staining or re-probing membranes with detection antibody in THBS2-competed membranes. To determine whether presence of THBS1 interfere with THBS2 ELISA, a 200ng/m1 of recombinant THBS1 protein was spiked into various concentration of recombinant THBS2 proteins (0 ng/ml to 20 ng/ml) or human plasma of wide range of THBS2 in THBS2 ELISA assay.

Immunostaining of THBS2 on Human Pancreatic Cancer Tissue

The pancreatic tumor tissue sections were obtained from US Biomax (cat #PA1002) and each tissue spot was individually examined by their own pathologists certified according to WHO published standardizations of diagnosis, classification and pathological grade. Incidental PanIN I-II tissue section was derived from the head and neck of pancreas of pancreatic periampullary cancer patient at FCCC and its histology was confirmed by a pathologist (Dr. Joseph Anderson) at FCCC. This tissue blocks do not correspond to plasma samples where plasma THBS2 concentrations were measured. The paraffin embedded tissues were antigen-retrieved by boiling in pH 6.0 Citric Acid buffer after de-paraffination. Next, the endogenous peroxidase activity in tissue slides was quenched in hydrogen peroxide solution for 15 min at RT. Tissues were blocked with avidin/biotin blocking (Vector lab, Burlingame, CA) for 15 min each, followed by non-protein blocker (Thermo Scientific) for 30 min at RT. Primary antibodies were applied and incubated for 12-16 hours at 4° C. Two primary antibodies for THBS2 were used for the current study: Goat polyclonal THBS2 antibody (dilution 1:25, sc-7655, Santa Cruz) and rabbit polyclonal THBS2 antibody (dilution 1:100, TA590658, Origene). It is not clear where TA590658 antibody recognize and whether it detects secreted THBS2. Yet, SC-7655 antibody can detect both secreted and cytoplasmic THBS2 since it targets the epitopes of 15-20 amino acids in length that are located within the first 50 amino acids of the peptide sequence for Thrombospondin-2, whose signal peptides are located between 1-18 amino acids. Only 2 amino acids of the epitope are overlapped to the signal peptides and the remaining are overlapped over the main body of the peptides. A peptide was available for sc-7655 from Santa Cruz, thus the SC-7655 antibodies were incubated with the corresponding peptides in 10-fold excess for 30 min prior to being applied onto tissue section to confirm the specificity of signals. Also, no primary antibody controls for sc-7655 and TA590658 antibodies were used for negative controls. After washing twice, tissues were incubated with biotinylated anti-goat IgG or rabbit IgG (Vector lab) at 37° C. for 30 min. Tissue sections were conjugated with avidin-Horseradish peroxidase (HRP) by using VectaStain Elite ABC kit (vector lab) at 37° C. for 30 min, followed by developing with DAB peroxidase substrate kit (Vector Lab) for peroxidase for 4-5 min. Developed tissue sections were stained with hematoxylin for nucleus, dehydrated, and mounted. The THBS2 sequence of the peptide was confirmed by mass spectrometry.

Statistical Analysis

The primary comparison for this study was defined as PDAC cases (all stages) vs. healthy controls. In order to explore any relationships between patient demographic information and THBS2 plasma concentration, a Spearman correlation coefficient was calculated for continuous variables (age) and Median expression concentrations were calculated for categorical variables (sex: Male vs Female, presence of diabetes mellitus: No vs. Yes). Based upon the data obtained from the current Phase 1 and 2 studies, no apparent associations between age, sex, diabetes mellitus, jaundice, and THBS2 was observed. Given this lack of association, any concerns regarding the potential for confounding were mitigated and these clinical factors were not included in subsequent multivariable modeling.

Univariate and multivariable logistic regression models were developed to consider each candidate biomarker (THBS2) alone and combined with CA19-9. The response variable was coded as 1 to indicate the presence of cancer, 0 for controls. Candidate biomarkers (THBS2) were entered as continuous variables. CA19-9 was dichotomized as 0=normal (<55 U/mL) or 1=elevated (≥55 U/mL). The area under the ROC curve (AUC) was calculated for each model considered. In order to asses if the difference observed between AUCs from the CA19-9 & THBS2 and the CA19-9 alone models was statistically significantly different from 0, a test statistic T (T=AUC_{Ca199}-AUC_{Ca199+THBS2})²/(S²_{Ca199}±S²_{Ca199+THBS2})(56) was considered, which looks at the difference in AUC between the two models divided by sum of the variances from the two models. The fact that this test statistic follows a chi-squared distribution with 1 degree of freedom under the null hypothesis was used to calculate a resulting p-value. A bootstrap percentile confidence interval approach was used to estimate a 95% confidence interval (CI) for the AUC. This approach re-sampled the dataset (1000 times) then ran the logistic regression models to calculate area under the ROC curve (AUC) on each bootstrapped dataset to approximate the sampling distribution of the AUC. The 2.5^{th} and 97.5^{th} percentiles from this distribution of AUC values will then be used as estimates of lower and upper bounds for the 95% CI for the AUC.

A similar approach was considered for each of the sub-analyses that stratified by stage (early stage, late stage) and other comparison groups (IPMN, chronic pancreatitis, or PNET). A Kappa statistic was calculated to assess agreement (below cutoff vs above cutoff) in the THBS2 assay results from each of the 2 independent labs in the cross-validation study. Analyses were performed using SAS 9.4 on Linux (SAS Institute, Cary N.C.).

### REFERENCES

1. L. Rahib et al., Projecting cancer incidence and deaths to 2030: the unexpected burden of thyroid, liver, and pancreas cancers in the United States. Cancer Res 74, 2913-2921 (2014).
2. R. L. Siegel, K. D. Miller, A. Jemal, Cancer statistics, 2016. CA Cancer J Clin 66, 7-30 (2016).
3. S. T. Chari et al., Early detection of sporadic pancreatic cancer: summative review. Pancreas 44, 693-712 (2015).
4. K. Satake, G. Kanazawa, I. Kho, Y. S. Chung, K. Umeyama, A clinical evaluation of carbohydrate antigen 19-9 and carcinoembryonic antigen in patients with pancreatic carcinoma. J Surg Oncol 29, 15-21 (1985).
5. M. J. Duffy et al., Tumor markers in pancreatic cancer: a European Group on Tumor Markers (EGTM) status report. Ann Oncol 21, 441-447 (2010).
6. G. Y. Locker et al., ASCO 2006 update of recommendations for the use of tumor markers in gastrointestinal cancer. J Clin Oncol 24, 5313-5327 (2006).
7. K. S. Goonetilleke, A. K. Siriwardena, Systematic review of carbohydrate antigen (CA 19-9) as a biochemical marker in the diagnosis of pancreatic cancer. Eur J Surg Oncol 33, 266-270 (2007).
8. M. A. Tempero et al., Relationship of carbohydrate antigen 19-9 and Lewis antigens in pancreatic cancer. Cancer Res 47, 5501-5503 (1987).
9. H. C. Harsha et al., A compendium of potential biomarkers of pancreatic cancer. PLoS Med 6, e1000046 (2009).
10. J. R. Mayers et al., Elevation of circulating branched-chain amino acids is an early event in human pancreatic adenocarcinoma development. Nat Med 20, 1193-1198 (2014).
11. S. A. Melo et al., Glypican-1 identifies cancer exosomes and detects early pancreatic cancer. Nature 523, 177-182 (2015).
12. K. H. Yu et al., Stable isotope dilution multidimensional liquid chromatography-tandem mass spectrometry for pancreatic cancer serum biomarker discovery. J Proteome Res 8, 1565-1576 (2009).
13. Chan et al., Validation of biomarkers that complement CA19.9 in detecting early pancreatic cancer. Clin Cancer Res 20, 5787-5795 (2014).
14. S. Makawita et al., Validation of four candidate pancreatic cancer serological biomarkers that improve the performance of CA19.9. BMC Cancer 13, 404 (2013).
15. T. P. Radon et al., Identification of a Three-Biomarker Panel in Urine for Early Detection of Pancreatic Adenocarcinoma. Clin Cancer Res 21, 3512-3521 (2015).
16. N. A. Schultz et al., MicroRNA biomarkers in whole blood for detection of pancreatic cancer. JAMA 311, 392-404 (2014).
17. M. Bauden et al., Circulating nucleosomes as epigenetic biomarkers in pancreatic cancer. Clin Epigenetics 7, 106 (2015).
18. M. E. Miller, S. L. Hui, W. M. Tierney, Validation techniques for logistic regression models. Stat Med 10, 1213-1226 (1991).
19. R. B. Rao, G. Fung, R. Rosales, An Experimental Evaluation. Proceedings of the 2008 SIAM International Conference on Data Mining, 588-598 (2008).
20. C. Jenkinson et al., Decreased Serum Thrombospondin-1 Levels in Pancreatic Cancer Patients Up to 24 Months Prior to Clinical Diagnosis: Association with Diabetes Mellitus. Clin Cancer Res 22, 1734-1743 (2016).
21. J. E. Minis et al., Cross-species antibody microarray interrogation identifies a 3-protein panel of plasma biomarkers for early diagnosis of pancreas cancer. Clin Cancer Res 21, 1764-1771 (2015).
22. B. M. Nolen et al., Prediagnostic serum biomarkers as early detection tools for pancreatic cancer in a large prospective cohort study. PLoS One 9, e94928 (2014).
23. M. Capello et al., Sequential Validation of Blood-Based Protein Biomarker Candidates for Early-Stage Pancreatic Cancer. J Natl Cancer Inst 109, (2017).
24. A. Maitra, R. H. Hruban, Pancreatic cancer. Annu Rev Pathol 3, 157-188 (2008).
25. J. Kim et al., An iPSC line from human pancreatic ductal adenocarcinoma undergoes early to invasive stages of pancreatic cancer progression. Cell Rep 3, 2088-2099 (2013).
26. N. Bardeesy et al., Smad4 is dispensable for normal pancreas development yet critical in progression and tumor biology of pancreas cancer. Genes Dev 20, 3130-3146 (2006).
27. S. Jones et al., Core signaling pathways in human pancreatic cancers revealed by global genomic analyses. Science 321, 1801-1806 (2008).
28. M. S. Pepe, Z. Feng, H. Janes, P. M. Bossuyt, J. D. Potter, Pivotal evaluation of the accuracy of a biomarker used for classification or prediction: standards for study design. J Natl Cancer Inst 100, 1432-1438 (2008).
29. M. S. Pepe et al., Phases of biomarker development for early detection of cancer. J Natl Cancer Inst 93, 1054-1061 (2001).
30. E. Kolker et al., MOPED: Model Organism Protein Expression Database. Nucleic Acids Res 40, D1093-1099 (2012).
31. V. Nanjappa et al., Plasma Proteome Database as a resource for proteomics research: 2014 update. Nucleic Acids Res 42, D959-965 (2014).
32. C. Wu et al., BioGPS: an extensible and customizable portal for querying and organizing gene annotation resources. Genome Biol 10, R130 (2009).
33. Prassas et al., False biomarker discovery due to reactivity of a commercial ELISA for CUZD1 with cancer antigen CA125. Clin Chem 60, 381-388 (2014).
34. K. Y. Bilimoria et al., Validation of the 6th edition AJCC Pancreatic Cancer Staging System: report from the National Cancer Database. Cancer 110, 738-744 (2007).
35. V. Molina et al., CA 19-9 in pancreatic cancer: retrospective evaluation of patients with suspicion of pancreatic cancer. Tumour Biol 33, 799-807 (2012).
36. L. Yan et al., Confounding effect of obstructive jaundice in the interpretation of proteomic plasma profiling data for pancreatic cancer. J Proteome Res 8, 142-148 (2009).
37. M. Wagner et al., Curative resection is the single most important factor determining outcome in patients with pancreatic adenocarcinoma. Br J Surg 91, 586-594 (2004).
38. S. Yachida et al., Distant metastasis occurs late during the genetic evolution of pancreatic cancer. Nature 467, 1114-1117 (2010).
39. D. J. Brat, K. D. Lillemoe, C. J. Yeo, P. B. Warfield, R. H. Hruban, Progression of pancreatic intraductal neoplasias to infiltrating adenocarcinoma of the pancreas. Am J Surg Pathol 22, 163-169 (1998).
40. S. Nie et al., Glycoprotein biomarker panel for pancreatic cancer discovered by quantitative proteomics analysis. J Proteome Res 13, 1873-1884 (2014).
41. P. Bornstein, S. Devarayalu, P. Li, C. M. Disteche, P. Framson, A second thrombospondin gene in the mouse is similar in organization to thrombospondin 1 but does not respond to serum. Proc Natl Acad Sci U S A 88, 8636-8640 (1991).
42. D. J. Good et al., A tumor suppressor-dependent inhibitor of angiogenesis is immunologically and functionally indistinguishable from a fragment of thrombospondin. Proc Natl Acad Sci U S A 87, 6624-6628 (1990).
43. T. Hawighorst et al., Thrombospondin-2 plays a protective role in multistep carcinogenesis: a novel host anti-tumor defense mechanism. EMBO J 20, 2631-2640 (2001).
44. R. Sun et al., Down regulation of Thrombospondin2 predicts poor prognosis in patients with gastric cancer. Mol Cancer 13, 225 (2014).
45. P. M. Bossuyt et al., The STARD statement for reporting studies of diagnostic accuracy: explanation and elaboration. Clin Chem 49, 7-18 (2003).
46. S. T. Chari et al., Probability of pancreatic cancer following diabetes: a population-based study. Gastroenterology 129, 504- 511 (2005).
47. G. M. Petersen, Familial Pancreatic Adenocarcinoma. Hematol Oncol Clin North Am 29, 641-653 (2015).
48. K. Rustgi, Familial pancreatic cancer: genetic advances. Genes Dev 28, 1-7 (2014).
49. G. Aggarwal, K. G. Rabe, G. M. Petersen, S. T. Chari, New-onset diabetes in pancreatic cancer: a study in the primary care setting. Pancreatology 12, 156-161 (2012).
50. R. R. McWilliams et al., Nucleotide excision repair pathway polymorphisms and pancreatic cancer risk: evidence for role of MNIS19L. Cancer Epidemiol Biomarkers Prey 18, 1295-1302 (2009).
51. W. J. Qian et al., Enhanced detection of low abundance human plasma proteins using a tandem IgY12-SuperMix immunoaffinity separation strategy. Mol Cell Proteomics 7, 1963-1973 (2008).
52. H. Chi et al., pFind-Alioth: A novel unrestricted database search algorithm to improve the interpretation of high-resolution MS/MS data. J Proteomics 125, 89-97 (2015).
53. E. Cerami et al., The cBio cancer genomics portal: an open platform for exploring multidimensional cancer genomics data. Cancer Discov 2, 401-404 (2012).
54. J. Gao et al., Integrative analysis of complex cancer genomics and clinical profiles using the cBioPortal. Sci Signal 6, pll (2013).
55. B. Li, C. N. Dewey, RSEM: accurate transcript quantification from RNA-Seq data with or without a reference genome. BMC Bioinformatics 12, 323 (2011).
56. Mithat Gonen, Analyzing Receiver Operating Characteristic Curves with SAS. (SAS Institute, 2007).

Various publications, patents and patent applications are cited herein, the contents of which are hereby incorporated by reference herein in their entireties.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method for diagnosing pancreatic cancer or a predisposition for developing pancreatic cancer in a subject, the method comprising determining the concentration of both a THBS2 protein and a CA19-9 protein in a biological sample obtained from the subject, wherein an increase in the combination of values of the concentration of both THBS2 protein and CA19-9 protein in the biological sample from the subject, as compared with a concentration cutoff value for independently THBS2 protein or CA19-9 protein, is an indication that the subject has a pancreatic cancer or a predisposition for developing a pancreatic cancer, wherein when the pancreatic cancer or the predisposition for developing the pancreatic cancer is detected in the subject, an anti-cancer treatment is recommended for the subject.

2. A method for determining the efficacy of an anti-cancer treatment for pancreatic cancer in a subject in need thereof, the method comprising determining the concentration of both a THBS2 protein and a CA19-9 protein in a biological sample obtained from the subject, wherein when the combination of values of the concentration of both THBS2 protein and CA19-9 protein in the biological sample from the subject is unchanged or lower as compared with a concentration cutoff value for independently THBS2 protein or CA19-9 protein, the treatment is efficacious, and when the treatment is not efficacious, an additional or a modified anti-cancer treatment is recommended for the subject.

3. The method of claim 1, wherein the concentration cutoff value for THBS2 protein is between about 30 to about 50 ng/ml.

4. The method of claim 3, wherein the concentration cutoff value for THBS2 protein is about 42 ng/ml.

5. The method of claim 1, wherein the concentration cutoff value for CA19-9 protein is between about 30 to about 1000 U/ml.

6. The method of claim 1, wherein the concentration cutoff value for CA19-9 protein is about 55 U/ml.

7. The method of claim 1, wherein the specificity of the combination of values is at least 90%.

8. The method of claim 1, wherein the specificity of the combination of values is at least 95%.

9. The method of claim 1, wherein the sensitivity of the combination of values is at least 80%.

10. The method of claim 1, wherein the sensitivity of the combination of values is at least 85%.

11. The method of claim 1, wherein the pancreatic cancer is a pancreatic ductal adenocarcinoma (PDAC).

12. The method of claim 1, wherein the anti-cancer treatment comprises at least one selected from the group consisting of surgical treatment, chemotherapy, radiation therapy, immunotherapy, gene therapy, biological modifier therapy, and cancer vaccine therapy.

13. The method of claim 1, wherein the biological sample is selected from the group consisting of a blood sample, a serum sample, a plasma sample, a stool sample, a urine sample, a pancreatic cyst fluid sample, and a tissue sample.

14. The method of claim 1, wherein the concentration of both THBS2 protein and CA19-9 protein is detected using a reagent that specifically binds to THBS2 protein and CA19-9 protein, respectively.

15. The method of claim 14, wherein the reagent is a monoclonal antibody or antigen-binding fragment thereof, or a polyclonal antibody or antigen-binding fragment thereof.

16. The method of claim 1, wherein the concentration of both THBS2 protein and CA19-9 protein is detected by enzyme-linked immunosorbent assay (ELISA).

17. The method of claim 1, wherein THBS2 protein comprises an amino acid sequence of SEQ ID NO: 1.

18. A method of determining whether a subject has pancreatic cancer, the method comprising:
a) detecting a THBS2 measurement in one or more biological samples obtained from the subject; and
b) comparing the detected THBS2 measurement to at least one of a THBS2 measurement from a healthy subject and a THBS2 cutoff value,
wherein an increase in the detected THBS2 measurement as compared to the healthy subject THBS2 measurement or the THBS2 cutoff value indicates that the subject has pancreatic cancer.

19. A method of determining whether a subject has pancreatic cancer, the method comprising:
a) detecting a THBS2 measurement and a CA19-9 measurement in one or more biological samples obtained from the subject;
b) comparing the detected THBS2 measurement to at least one of a THBS2 measurement from a healthy subject and a THBS2 cutoff value; and
c) comparing the detected CA19-9 measurement to a CA19-9 cutoff value,
wherein an increase in the THBS2 measurement as compared to the healthy subject THBS2 measurement or the THBS2 cutoff value and/or an increase in the detected CA19-9 measurement as compared to the CA19-9 cutoff value indicates that the subject has pancreatic cancer.

20. The method of claim 1, wherein when pancreatic cancer is detected in the subject, an anti-cancer treatment is recommended for the subject.

21. The method of claim 1, the pancreatic cancer is an early stage pancreatic cancer.

22. The method of claim 1, the pancreatic cancer is a resectable pancreatic cancer.

23. The method of claim 1, wherein the subject is human.

24. The method of claim 1, wherein the biological sample is selected from the group consisting of a blood sample, a serum sample, a plasma sample, a stool sample, a urine sample, a pancreatic cyst fluid sample, and a tissue sample.

25. The method of claim 1, wherein the THBS2 is a THBS2 protein.

26. The method of claim 25, wherein the level of THBS2 protein is detected using a reagent which specifically binds to the THBS2 protein.

27. The method of claim 26, wherein the reagent is a monoclonal antibody or antigen-binding fragment thereof, or a polyclonal antibody or antigen-binding fragment thereof.

28. The method of claim 25, wherein the level of the THBS2 protein is detected by enzyme-linked immunosorbent assay (ELISA).

29. The method of claim 19, wherein the THBS2 comprises a transcribed polynucleotide of THBS2 or portion thereof

30. The method of claim 19, wherein the CA19-9 is a CA19-9 protein.

31. The method of claim 30, wherein the level of CA19-9 protein is detected using a reagent which specifically binds to the CA19-9 protein.

32. The method of claim 31, wherein the reagent is a monoclonal antibody or antigen-binding fragment thereof, or a polyclonal antibody or antigen-binding fragment thereof.

33. The method of claim 30, wherein the level of the CA19-9 protein is detected by enzyme-linked immunosorbent assay (ELISA).

34. The method of claim 19, wherein the THBS2 cutoff value is a THBS2 protein cutoff value of between about 30 to about 50 ng/ml.

35. The method of claim 34, wherein the THBS2 protein cutoff value is about 42 ng/ml.

36. The method of claim 19, wherein the CA19-9 cutoff value is between about 30 to about 1000 U/ml.

37. The method of claim 19, wherein the CA19-9 cutoff value is a CA19-9 protein cutoff value of about 55 U/ml.

38. A kit for diagnosing whether a subject has pancreatic cancer, comprising reagents useful for detecting THBS2 in one or more biological samples obtained from the subject.

39. A kit for diagnosing whether a subject has pancreatic cancer, comprising reagents useful for detecting THBS2 and CA19-9 in one or more biological samples obtained from the subject.

40. The kit of claim 39, comprising one or more of packaged probe and primer sets, arrays/microarrays, biomarker-specific antibodies or beads for detecting the THBS2 and/or CA19-9.

41. The kit of claim 39, comprising at least one monoclonal antibody or antigen-binding fragment thereof, or a polyclonal antibody or antigen-binding fragment thereof, for detecting the THBS2 and/or CA19-9.

42. The kit of claim 39, wherein the pancreatic cancer is an early stage pancreatic cancer.

43. The kit of claim 39, wherein the pancreatic cancer is a resectable pancreatic cancer or a pre-cancerous lesion.

44. The kit of claim 39, wherein the subject is human.

45. The kit of claim 39, wherein the biological sample is a blood sample.

46. The kit of claim 38, further comprising an instruction describing that an increase in the level of the THBS2 as compared to a THBS2 cutoff value indicates that the subject has pancreatic cancer.

47. The kit of claim 39, further comprising an instruction describing that an increase in the level of the THBS2 as compared to a THBS2 cutoff value and/or an increase in the level of the CA19-9 as compared to a CA19-9 cutoff value indicates that the subject has pancreatic cancer.

48. The kit of claim 47, wherein the THBS2 cutoff value is a THBS2 protein cutoff value of between about 30 to about 50 ng/ml.

49. The kit of claim 47, wherein the THBS2 protein cutoff value is about 42 ng/ml.

50. The kit of claim 47, wherein the CA19-9 cutoff value is between about 30 to about 1000 U/ml.

51. The kit of claim 47, wherein the CA19-9 cutoff value is a CA19-9 protein cutoff value of about 55 U/ml.

52. A method for determining a cutoff value of THBS2 for diagnosis of pancreatic cancer in a subject, the method comprising:
a) measuring the distribution of THBS2 values in a group of healthy subjects;
b) selecting a THBS2 value wherein the selected THBS2 value has a false positive rate of between about 0 to about 5%; and
c) measuring the sensitivity value and specificity value of the selected THBS2 value in detecting pancreatic cancer in a group of subjects having pancreatic cancer,
wherein the sensitivity value of at least about 50% and the specificity value of at least about 90% indicate the selected THBS2 value is a cutoff value of THBS2 for diagnosis of pancreatic cancer in a subject.

53. The method of claim 52, wherein the false positive rate is about 1%.

54. The method of claim 52, wherein the sensitivity value is about 50%.

55. The method of claim 52, wherein the specificity value is about 99%.

56. (canceled)

57. (canceled)

58. A method of managing a subject suspected of having pancreatic cancer, the method comprising:
a) detecting a THBS2 measurement in one or more biological samples from the subject; and
b) comparing the detected THBS2 measurement to at least one of a THBS2 measurement from a healthy subject and/or a THBS2 cutoff value,
wherein if an increase in the detected THBS2 measurement as compared to the healthy subject THBS2 measurement or the THBS2 cutoff value is detected, an imaging evaluation is performed to detect pancreatic cancer,
wherein if the pancreatic cancer is confirmed by the imaging evaluation, a biopsy of the pancreatic cancer is performed on the subject, followed by an anti-cancer treatment of the subject.

59. A method of managing a subject suspected of having pancreatic cancer, the method comprising:
a) detecting a THBS2 measurement in one or more biological samples obtained from the subject; and
b) comparing the detected THBS2 measurement to at least one of a THBS2 measurement from a healthy subject and/or a THBS2 cutoff value,
wherein if an increase in the detected THBS2 measurement as compared to the healthy subject THBS2 measurement or the THBS2 cutoff value is not detected, follow-up screening/surveillance is performed to monitor the subject for early stage pancreatic cancer.

60. A method of managing a subject suspected of having pancreatic cancer, the method comprising:
a) detecting a THBS2 measurement and a CA19-9 measurement in the one or more biological samples obtained from the subject;
b) comparing the detected THBS2 measurement to at least one of a THBS2 measurement from a healthy subject and a THBS2 cutoff value; and
c) comparing the detected CA19-9 measurement to a CA19-9 cutoff value,
wherein if an increase in the THBS2 measurement as compared to the healthy subject THBS2 measurement or the THBS2 cutoff value and/or an increase in the detected CA19-9 measurement as compared to the CA19-9 cutoff value is detected, an imaging evaluation is performed to detect pancreatic cancer,
wherein if the pancreatic cancer is confirmed by the imaging evaluation, a biopsy of the pancreatic cancer is performed on the subject, followed by an anti-cancer treatment of the subject.

61. A method of managing a subject suspected of having pancreatic cancer, the method comprising:
a) detecting a THBS2 measurement and a CA19-9 measurement in the one or more biological samples obtained from the subject;
b) comparing the detected THBS2 measurement to at least one of a THBS2 measurement from a healthy subject and a THBS2 cutoff value; and
c) comparing the detected CA19-9 measurement to a CA19-9 cutoff value,
wherein if neither an increase in the THBS2 measurement as compared to the healthy subject THBS2 measurement or the THBS2 cutoff value nor an increase in the detected CA19-9 measurement as compared to the CA19-9 cutoff value is detected, a follow-up screening/surveillance is performed to monitor the subject for early stage pancreatic cancer.

62. A method for treating pancreatic cancer in a subject, the method comprising:
(a) detecting a combination of values in the concentration of both THBS2 protein and CA19-9 protein, in a biological sample obtained from the subject,
(b) comparing the combination of values in the concentration of both THBS2 protein and CA19-9 protein in the biological sample, with a concentration cutoff value for independently THBS2 protein or CA19-9 protein, and
(c) when the combination of values in the concentration of both THBS2 protein and CA19-9 protein in the biological sample is higher than the concentration cutoff value for independently THBS2 protein or CA19-9 protein, the subject is administered a chemotherapy, a radiation therapy, an immunotherapy, a gene therapy, a biological modifier therapy, or a cancer vaccine therapy to the subject, wherein the number of pancreatic cancer cells within the subject is reduced.

63. A method for treating pancreatic cancer in a subject, the method comprising:
(a) detecting a combination of values in the concentration of both THBS2 protein and CA19-9 protein in a biological sample obtained from the subject,
(b) comparing the combination of values in the concentration of both THBS2 protein and CA19-9 protein in the biological sample, with a concentration cutoff value for independently THBS2 protein or CA19-9 protein, and
(c) when the combination of values in the concentration of both THBS2 protein and CA19-9 protein in the biological sample is higher than the concentration cutoff value for independently THBS2 protein or CA19-9 protein, the pancreatic cancer cells are surgically removed from the subject.

64. The method of claim 63, wherein the subject is a human.

65. The method of claim 63, wherein the pancreatic cancer is an early stage pancreatic cancer or a resectable pancreatic cancer.

66. The method of claim 63, wherein the cutoff value for THBS2 protein is between about 30 to about 50 ng/ml.

67. The method of claim 66, wherein the cutoff value for THBS2 protein is about 42 ng/ml.

68. The method of claim 63, wherein the cutoff value for CA19-9 protein is between about 30 to about 1000 U/ml.

69. The method of claim 68, wherein the cutoff value for CA19-9 protein is about 55 U/ml.

70. A method for treating pancreatic cancer in a subject, the method comprising administering a chemotherapy, a radiation therapy, an immunotherapy, a gene therapy, a biological modifier therapy, or a cancer vaccine therapy to a subject identified as having pancreatic cancer and a combination of values in the concentration of both THBS2 protein and CA19-9 protein in a biological sample obtained from the subject, wherein the combination of values in the concentration of both THBS2 protein and CA19-9 protein in the biological sample is higher than a concentration cutoff value for independently THBS2 protein or CA19-9 protein, and wherein the number of pancreatic cancer cells within the subject is reduced.

71. A method for treating pancreatic cancer in a subject, the method comprising surgically removing pancreatic cancer cells from a subject identified as having pancreatic cancer and a combination of values in the concentration of both THBS2 protein and CA19-9 protein in a biological sample obtained from the subject, wherein the combination of values in the concentration of both THBS2 protein and CA19-9 protein in the biological sample is higher than a concentration cutoff value for independently THBS2 protein or CA19-9 protein.

72. The method of claim 70, wherein the subject is a human.

73. The method of claim 70, wherein the pancreatic cancer is an early stage pancreatic cancer or a resectable pancreatic cancer.

74. The method of claim 70, wherein the cutoff value for THBS2 protein is between about 30 to about 50 ng/ml.

75. The method of claim 74, wherein the cutoff value for THBS2 protein is about 42 ng/ml.

76. The method of claim 70, wherein the cutoff value for CA19-9 protein is between about 30 to about 1000 U/ml.

77. The method of claim 76, wherein the cutoff value for CA19-9 protein is about 55 U/ml.
